(19) United States
(12) Patent Application Publication
(10) Pub. No.: US 2024/0060078 A1
(43) Pub. Date: Feb. 22, 2024

(54) NOVEL MOGROSIDE PRODUCTION SYSTEM AND METHODS

(71) Applicant: The Coca-Cola Company, Atlanta, GA (US)

(72) Inventors: Alec J. HAYES, Marietta, GA (US); Christopher P. MERCOGLIANO, Atlanta, GA (US); Tengfang HUANG, Cary, NC (US); Fayaz KHAZI, Chapel Hill, NC (US)

(73) Assignee: The Coca-Cola Company, Atlanta, GA (US)

(21) Appl. No.: 17/906,611

(22) PCT Filed: Mar. 17, 2021
(86) PCT No.: PCT/US2021/022803

§ 371 Date: Sep. 16, 2022

Related U.S. Application Data
(60) Provisional application No. 62/990,802, filed on Mar. 17, 2020.

(51) Int. Cl. C12N 15/82; A23L 27/30; C12N 9/02; C12N 9/10; C12N 9/14; C12N 9/90; C12N 15/52

(52) U.S. Cl. CPC C12N 15/8243; A23L 27/36; C12N 9/0071; C12N 9/1051; C12N 9/14; C12N 9/90; C12N 15/52; C12Y 504/99033

(57) ABSTRACT

The present disclosure presents a solution to producing mogrosides and mogroside-based sweeteners having low or no calorie. By using recombinant gene and plant transformation techniques, non-native genes encoding mogroside pathway enzymes are introduced/implemented into the genome of a plant hereby forming a transgenic plant, wherein the plant by the native genome thereof prior to transformation may not produce mogrosides naturally. Such transgenic plant and a progeny thereof are enabled to produce non-native mogrol precursors, mogrol, mogrosides, and/or metabolites or derivatives thereof.

This application is being filed on Mar. 17, 2021, as a PCT International Patent application and claims the benefit of and priority to U.S. Provisional patent application Ser. No. 62/990,802, filed Mar. 17, 2020, the entire disclosure of which is incorporated by reference in its entirety.

Pursuant to 37 C.F.R. § 1.821(c) or (e), a file containing an ASCII text version of the Sequence Listing has been submitted concomitant with this application, the contents of which are hereby incorporated by reference.

### INTRODUCTION

With the increased awareness of healthy diets and potential risks of obesity and diabetes in global countries, low or non-calorie sweetener alternative to traditional high calorie sweeteners are becoming increasingly important to the food and beverage business as well as other industries. These alternative natural sweeteners are used to substitute artificial sweeteners and the high calorie sweeteners comprising sucrose, fructose, and glucose. Like some artificial sweeteners, some provide a greater sweetening effect than comparable amounts of caloric sweeteners; thus, smaller amounts of these alternatives are required to achieve sweetness comparable to that of sugar. However, some low calorie sweeteners can be expensive to produce and/or possess unfavorable taste characteristics and/or off-tastes, including but not limited to sweetness linger, delayed sweetness onset, negative mouth feels, bitter, metallic, cooling, astringent, and licorice-like tastes.

A few natural plants biosynthetically produce low or non-calorie sweeteners. For example, mogrosides, as an important class of natural sweeteners, are chemically a class of triterpene glycosides or mogrol glycosides naturally produced by Monk Fruit (scientific name: Siraitia grosvenorii). Mogrosides contain zero calorie, and are 100-400 times sweeter than sucrose. Mogrosides have also been reported to have a variety of important pharmacological effects.

Mogrosides are highly stable molecules based on a triterpene skeleton, formed of varying numbers of glucose units, from 1 to 6 attached to carbon 24 and/or carbon 3 (FIG. 1) of the triterpene backbone. Various mogrosides and their structures are shown in FIG. 2. Mogrosides may also comprise non-glucose moiety such as grosmomoside I.

In general, the natural biosynthesis of mogrosides are only available in Siraitia grosvenorii. Both the fresh and dried Siraitia grosvenorii are extracted to yield a powder that is about 80% mogrosides, wherein, the main component is Mogroside V. The biosynthetic pathways for mogrol/mogrosides and all the enzymes involved in the steps from squalene to Mogroside V have been identified by genome sequencing and transcriptome analysis at each developmental stage of the fruit. FIG. 1 shows the Mogroside V biosynthetic pathway (Seki et al Bioscience, Biotechnology, and Biochemistry, 2018 VOL. 82, NO. 6, 927-934).

Although plants like Siraitia grosvenorii make natural low and non-calorie sweeteners, production of sweeteners from these plants is limited due to the limited natural or agricultural production of these plants. More importantly, sweeteners produced in plants, as opposed to chemical or biochemical synthesis in vitro, tend to be more acceptable by consumers. Also sweeteners produced in plants could be of use for a variety of reasons. Such fruits or plants could be used to produce not only low and non-calorie sweetener, but also flavors, extracts, or juices, including filler juices for beverages, for use in foods and beverages with reduced calorie and other nutritional benefits.

In addition, mass production of mogrosides in vitro or in microorganisms, although conceptually proved, may require extensive processing and thus not economically advantageous. The biosynthetic pathways for producing mogrosides have been tried in microorganisms for fermentation. However, small sweetening molecules such as mogrosides have not been fully developed.

US 2019/0071705 to Patron provides a method for produce Mogroside IIIE in a recombinant host cell comprising cultivating the recombinant host cell in a culture medium under certain conditions, wherein the gene of the recombinant host cell express enzymes which catalyze production of Mogroside IIIE

WO2018/229283 to Houghton-Larson provides a recombinant host cell capable of producing one or more mogroside compounds in a cell culture, wherein the host cell comprising a recombinant gene encoding a heterologous or an endogenous polypeptide capable of catalyzing production of mogrosides.

WO 2016/038617 to Itkin relates to methods of biosynthetically making and isolating mogroside-producing enzymes and methods of making mogrol precursors, mogrol, and mogrosides in recombinant host cells.

U.S. Pat. Nos. 9,932,619 and 9,920,349 both to Liu relate to in vitro methods and materials for enzymatic synthesis of mogroside compounds, and to methods of producing mogrol using cytochrome P450 enzymes and glycosylating mogrol using Uridine-5′-diphospho (UDP) dependent glucosyltransferases (UGTs) to produce various mogroside compounds.

Thus, there is a need for new methods and biological systems for efficient production of mogrosides, and it is against the above background that the present disclosure presents advantages and advancements to address this need.

### Novel Mogroside Production System and Methods

### SUMMARY OF DISCLOSURE

The present disclosure presents a solution to producing mogrol, mogrosides, and mogroside-based sweeteners having low or no calorie. By using recombinant gene and plant transformation techniques, non-native genes encoding morgol-producing and mogroside-producing enzymes are introduced/implemented into the genome of a natural plant thereby forming a transgenic plant, wherein the natural plant by the native genome thereof prior to transformation may not produce mogrol or mogrosides naturally. Such transgenic plant is enabled to produce non-native mogrol precursors, mogrol, mogrosides, and/or metabolites or derivatives thereof.

The provided solution is of significant advantage. First, the production of mogrol, low or non-calorie mogrosides via cultivation and reproduction of transgenic plants may have better techno-economics because of mature agricultural technologies. In addition, the solution may allow for mogroside production throughout more parts of the plant and not just within fruits, thereby enhancing the entire nutritional and economic value of the transgenic plant. Moreover, implementing mogrol- and/or mogroside-producing transgenes into fast-growing or fast-maturing plants/crops may improve efficiencies of mogroside production and processing, and provide cost-effective benefits. The provided solution may allow for novel low- or non-calorie foods and beverages by incorporation of these transgenic plants and materials or parts thereof.

It should be noted that previous disclosures primarily focused on genetically engineered microorganism (mostly yeast) based method for making mogrosides. The present disclosure distinctly describes a transgenic plant that is enabled to produce mogrol/mogrosides. Yeast-based methods are conceptually capable of synthesizing mogroside, but with little or no economic benefit. More importantly, sweeteners and food products derived from plants perceivably improve consumer acceptance. The transgenic plants according to the present disclosure allow for the production of juice or plant extracts or plant materials or other derived consumables with a lower ratio of calories to sweetness that could be used with less processing or with preferred additional flavor characteristics and profiles.

It is important to note that the ability of a transgenic organism comprising mogroside-producing transgenes to produce fruits and/or seeds is rare. Surprisingly, the transgenic plants according to the present disclosure produced various tissues including fruits and seeds, wherein the various tissues including fruits and seeds all comprise mogrosides. The mogrosides containing fruit of the present transgenic plants can be used as a source for various food and beverage products and therefore provides techno-economic advantages in food and consumable industry. In addition, the seed-producing transgenic plants of the present disclosure can benefit mass and cost-effective production of mogrosides by propagation of the seeds and agricultural reproduction of the transgenic plants using various plant-breeding technologies.

In one example application, watermelon fruit has great potential for production of low- and/or non-caloric sweeteners due to its large size and popular flavor. To design a genome editing or cis-genic strategy for pathway engineering, it is critical to identify watermelon fruit specific promoters that enables optimal expression of genetic payloads such as mogrosides producing sequences. Identification of these promoters requires a high-resolution transcriptomic dataset, from which a list of genes that are specifically expressed in the edible portion of watermelon fruit can be generated. The methods and systems described in the present disclosure advantageously provide an effective approach for tissue-specific expression of genes of interest at different developmental stage.

The present disclosure generally describes transgenic plants and biosynthetic systems thereof for making mogrol- and/or mogroside-producing enzymes and mogrol/mogrosides in the transgenic plants and tissues or parts thereof, and methods for making such transgenic plants.

In some embodiments, the present disclosure relates to a transgenic plant comprising a genomic transformation event, wherein the genomic transformation event produces a non-native expression or concentration of mogrol- and/or mogroside-producing enzyme(s), wherein the transgenic plant biosynthetically produces non-native mogrol precursors, mogrol, mogrosides, and/or metabolites or derivatives thereof. In certain embodiments of such transgenic plant, the genomic transformation event comprises an expression cassette, wherein the expression cassette comprises one or more of the nucleotide sequences as set forth in SEQ ID NOs: 1-31. In other embodiments, such expression cassette comprises one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

In other embodiments, the present disclosure relates to a transgenic plant comprising non-native mogrol precursors and/or mogrol, wherein the transgenic plant biosynthetically produces mogrol, mogrosides, and/or metabolites or derivatives thereof. In certain embodiments, such transgenic plant comprises an expression cassette, wherein the expression cassette comprises one or more of the nucleotide sequences as set forth in SEQ ID NOs: 1-31. In other embodiments, such expression cassette comprises one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

In certain embodiments, the transgenic plant of the present disclosure contains an obtainable part thereof, including but not limited to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the transgenic plant, wherein the part comprises mogrol precursors, mogrol, mogroside, and/or metabolites or derivatives thereof.

In other embodiments, the transgenic plant of the present disclosure is cultivatable and reproducible. A progeny or an ancestor of the transgenic plant is a source of non-native enzyme(s) enabling the progeny and the ancestor to produce mogrol, mogrosides, and/or metabolites or derivatives thereof. Propagation of the seed of the transgenic plant results in viable progeny thereof, wherein the progeny produces mogrol, mogrosides, and/or metabolites or derivatives thereof.

In some embodiments, the transgenic plant is a diploid plant. In some embodiments, the transgenic plant is a Cucurbitaceae/Curcubits. In certain embodiments, the transgenic plant is a transgenic watermelon (Citrullus lanatus).

The present disclosure also relates to food or beverage products obtainable from the transgenic plant, wherein the food or the beverage products contain mogrol, mogrosides, or mogroside-based sweeteners. In some embodiments, the present disclosure relates to mogroside-based sweetener, wherein the mogroside-based sweetener is extracted or purified from the transgenic plant or the part thereof according to the present disclosure. In certain embodiments, the methods for extracting and/or purifying mogroside-based sweeteners from the transgenic plants are steeping, chromatography, or absorption chromatogram.

The present disclosure relates to a method for making a transgenic plant producing non-native mogrosides, wherein the method comprises combining a plant with a genomic transformation event thereby forming the transgenic plant, wherein the genomic transformation event produces a non-native expression or concentration of mogrol-producing and/or mogroside-producing enzyme(s). Combining the plant with the genomic transformation event is generally performed using one or more of the following methods: use of liposomes, use of electroporation, use of chemicals that increase free DNA uptake, use of injection of the DNA directly into the plant, use of particle gun bombardment, use of transformation using viruses or pollen, use of microprojection, or use of Agrobacterium-mediated transformation.

In some embodiments, the present disclosure relates to a biosynthetic method for producing non-native mogrol precursors, mogrol, and mogrosides in a transgenic plant, comprising the steps of: (a) combining a plant with a genomic transformation event thereby forming a transgenic plant, wherein the genomic transformation event produces a non-native expression or concentration of mogrol-producing and/or mogroside-producing enzyme(s); (b) growing and regenerating a population of the transgenic plant; (c) selecting the transgenic plants that produce mogrosides; and (d) harvesting mogrosides. In certain embodiments, the biosynthetic method further comprises: preparing/providing plasmids comprising an expression cassette wherein the expression cassette expresses non-native mogrol-producing and/or mogroside-producing enzyme(s); transforming a host cell with the plasmids; and transfecting the plant with a plurality of the transformed host cell.

### Definition and Interpretation of Terms

The following definitions or interpretations of technical terms will be used throughout the present disclosure. The technical terms used herein are generally to be given the meaning commonly applied to them in the pertinent art of plant biology, molecular biology, bioinformatics, and plant breeding. All of the following term definitions apply to the complete content of this application. It is to be understood that as used in the specification and in the claims, “a” or “an” can mean one or more, depending upon the context in which it is used. Thus, for example, reference to “a cell” can mean that at least one cell can be utilized.

The term “essentially,” “about,” “approximately” and the like in connection with an attribute or a value, particularly also define exactly the attribute or exactly the value, respectively. The term “about” in the context of a given numeric value or range relates in particular to a value or range that is within 20%, within 10%, or within 5% of the value or range given. As used herein, the term “comprising” also encompasses the term “consisting of.”

The terms “peptides,” “oligopeptides,” “polypeptide,” “protein”, or “enzyme” are used interchangeably herein and refer to amino acids in a polymeric form of any length, linked together by peptide bonds, unless mentioned herein otherwise.

The terms “gene sequence(s),” “polynucleotide(s),” “nucleic acid sequence(s),” “nucleotide sequence(s),” “nucleic acid(s),” “nucleic acid molecule” are used interchangeably herein and refer to nucleotides, either ribonucleotides or deoxyribonucleotides or a combination of both, in a polymeric unbranched form of any length.

Transgenic/Transgene/Recombinant Gene

For the purposes of the present disclosure, “transgenic,” “transgene,” or “recombinant” means with regard to, for example, a nucleic acid sequence, an expression cassette, genetic construct, or a vector comprising the nucleic acid sequence or an organism transformed with the nucleic acid sequences, expression cassettes or vectors according to the disclosure, all those constructions brought about by recombinant methods in which either (a) the sequences of the nucleic acids or a part thereof, or (b) genetic control sequence(s) which is operably linked with the nucleic acid sequence according to the disclosure, for example a promoter, or (c) combinations of (a) and (b), are not located in their natural genetic environment or have been modified by recombinant methods e.g. modified and/or inserted artificially by genetic engineering methods.

As used herein, the term “transgenic” relates to an organism e.g. transgenic plant refers to an organism, e.g., a plant, plant cell, callus, plant tissue, or plant part that exogenously contains the nucleic acid, construct, vector, or expression cassette described herein or a part thereof which is preferably introduced by processes that are not essentially biological, preferably by Agrobacteria-mediated transformation or particle bombardment. A transgenic plant for the purposes of the present disclosure is thus understood as meaning, as above, that the nucleic acids described herein are not present in, or not originating from the genome of said plant, or are present in the genome of said plant but not at their natural genetic environment in the genome of said plant, it being possible for the nucleic acids to be expressed homologously or heterologously. However, as mentioned, transgenic also means that, while the nucleic acids according to the disclosure or used in the disclosed method are at their natural position in the genome of a plant, the sequence has been modified with regard to the natural sequence, and/or that the regulatory sequences of the natural sequences have been modified. Transgenic is preferably understood as meaning that the expression of naturally in that plant occurring nucleic acid sequences at an unnatural genetic environment in the genome, i.e. homologous expression, or that heterologous expression of not naturally in that plant occurring nucleic acid sequences takes place.

Plant/Transgenic Plant/Natural Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

A transgenic plant herein refers to a plant into which one or more transgenes from another species have been introduced into the genome of the plant, using genetic engineering techniques. The introduced transgene encodes and expresses non-native proteins or enzymes thereby enabling the transgenic plant to possess new characteristics such as producing non-native enzymatic pathway products that are not naturally present in the plant prior to introduction of the transgene. A transgenic plant is opposed to a natural plant, which is a product of nature, without artificial interference by human. A natural plant according to the present application refers to a wild-type plant, a plant that is not genetically modified by human, or an untransformed/non-transformed plant used as a control characterizing the transgenic plants made according to the present disclosure.

Endogenous/Native

An “endogenous” or “native” nucleic acid and/or a protein refers to the a nucleic acid and/or a protein in question as found in a plant in its natural form (i.e., without there being any human intervention like recombinant DNA engineering technology), but also refers to that same gene (or a substantially homologous nucleic acid/gene) in an isolated form subsequently (re)introduced into a plant (a transgene). A transgenic plant containing such a transgene may or may not encounter a substantial reduction of the transgene expression and/or substantial reduction of expression of the endogenous gene.

Exogenous

The term “exogenous” (in contrast to “endogenous”) nucleic acid or gene refers to a nucleic acid that has been introduced in a plant by means of recombinant DNA technology. An “exogenous” nucleic acid can either not occur in a plant in its natural form, be different from the nucleic acid in question as found in a plant in its natural form, or can be identical to a nucleic acid found in a plant in its natural form, but integrated not within its natural genetic environment. The corresponding meaning of “exogenous” is applied in the context of protein expression. For example, a transgenic plant containing a transgene, i.e., an exogenous nucleic acid, may, when compared to the expression of the endogenous gene, encounter a substantial increase of the expression of the respective gene or protein in total. A transgenic plant according to the present disclosure includes one or more exogenous nucleic acids integrated at any genetic loci and optionally the plant may also include the endogenous gene within the natural genetic background.

Expression Cassette

“Expression cassette” as used herein is a vector DNA capable of being expressed in a host cell. The DNA, part of the DNA or the arrangement of the genetic elements forming the expression cassette can be artificial. The skilled artisan is aware of the genetic elements that must be present in the expression cassette in order to be successfully yield expression. The expression cassette comprises a sequence of interest to be expressed operably linked to one or more control sequences (at least to a promoter) as described herein. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section for increased expression/overexpression. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The expression cassette may be integrated into the genome of a host cell and replicated together with the genome of said host cell.

Vector

Vector or vector construct is DNA (such as but, not limited to plasmids, viral DNA, and chromosome vector) artificial in part or total or artificial in the arrangement of the genetic elements contained-capable of replication in a host cell and used for introduction of a DNA sequence of interest into a host cell or host organism. A vector may be a construct or may comprise at least one construct. A vector may replicate without integrating into the genome of a host cell, e.g. a plasmid vector in a bacterial host cell, or it may integrate part or all of its DNA into the genome of the host cell and thus lead to replication and expression of its DNA. Host cells of the invention may be any cell selected from bacterial cells, such as Escherichia coli or Agrobacterium species cells, yeast cells, fungal, algal or cyanobacterial cells, or plant cells. The skilled artisan is aware of the genetic elements that must be present on the genetic construct in order to successfully transform, select and propagate host cells containing the sequence of interest. Typically the vector comprises at least one expression cassette. The one or more sequence(s) of interest is operably linked to one or more control sequences (at least to a promoter) as described herein. Additional regulatory elements may include transcriptional as well as translational enhancers. Those skilled in the art will be aware of terminator and enhancer sequences that may be suitable for use in performing the herein disclosed techniques.

Operably Linked

The term “operably linked” or “functionally linked” is used interchangeably and, as used herein, refers to a functional linkage between the promoter sequence and the gene of interest, such that the promoter sequence is able to direct transcription of the gene of interest.

Promoter/Plant Promoter/Strong Promoter/Weak Promoter

A “promoter” or “plant promoter” comprises regulatory elements, which mediate the expression of a coding sequence segment in plant cells. The “plant promoter” can originate from a plant cell, e.g. from the plant which is transformed with the nucleic acid sequence to be expressed in the present systems and described herein. This also applies to other “plant” regulatory signals, such as plant terminators. The promoters upstream of the nucleotide sequences useful in the methods of the present disclosure can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described herein, be linked operably to or comprise a suitable promoter which expresses the gene at the right point in time and with the required spatial expression pattern.

The promoter used herein broadly encompasses constitutive promoter, ubiquitous promoter, developmentally-regulated promoter, inducible promoter, organ-specific promoter, tissue-specific promoter, seed-specific promoter, green-tissue specific promoter, meristem-specific promoter, etc. A “ubiquitous promoter” is active in substantially all tissues or cells of an organism.

For the identification of functionally equivalent promoters, the promoter strength and/or expression pattern of a candidate promoter may be analyzed for example by operably linking the promoter to a reporter gene and assaying the expression level and pattern of the reporter gene in various tissues of the plant. Generally, by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter.

Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

Reporter Gene

“Selectable marker,” “selectable marker gene,” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics such as kanamycin (KAN) or hygromycin (Hyg). Expression of visual marker genes results in the formation of fluorescence (Green Fluorescent Protein, GFP; Red Fluorescent Protein, RFP; and derivatives thereof). This list represents only a small number of possible markers. A skilled artisan is familiar with such markers. Different markers are preferred, depending on the organism and the selection method.

Expression/Gene Expression

The term “expression” or “gene expression” means the transcription of a specific gene or specific genes or specific genetic construct. The term “expression” or “gene expression” in particular means the translation of the RNA and therewith the synthesis of the encoded protein/enzyme, i.e., protein/enzyme expression.

Percent Identity/Homology

As used herein, sequence identity, homology, or “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100.

Introduction/Implementation/Transformation

The term “introduction,” “implementation,” or “transformation” as referred to herein encompasses the transfer of an exogenous polynucleotide into a host cell, irrespective of the method used for transfer.

Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. Alternatively, a plant cell that cannot be regenerated into a plant may be chosen as host cell, i.e. the resulting transformed plant cell does not have the capacity to regenerate into a (whole) plant.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Ploidy/Ploidy Level/Chromosomal Ploidy/Polyploidy

Ploidy or chromosomal ploidy refers the number of complete sets of chromosomes occurring in the nucleus of a cell. Somatic cells, tissues, and individual organisms can be described according to the number of sets of chromosomes present (the “ploidy level”): monoploid (1 set), diploid (2 sets), triploid (3 sets), tetraploid (4 sets), pentaploid (5 sets), hexaploid (6 sets), heptaploid or septaploid (7 sets), etc. The generic term polyploidy is used herein to describe cells with three or more chromosome sets.

Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. For the purposes of this application, the original unmodulated expression may also be absence of any expression. The term “modulating the activity” or the term “modulating expression” shall mean any change of the expression of the target nucleic acid sequences and/or encoded proteins, which leads to increased or decreased yield-related trait(s) such as but not limited to increased or decreased seed yield and/or Increased or decreased growth of the plants. The expression can increase from zero (absence of, or immeasurable expression) to a certain amount, or can decrease from a certain amount to immeasurable small amounts or zero.

Generally, after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described herein.

Following DNA transfer and regeneration, putatively transformed plants may also be evaluated, for the presence of the gene of interest, copy number and/or genomic organization.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Throughout this application a plant, plant part, seed or plant cell transformed with, or interchangeably transformed by, a construct or transformed with or by a nucleic acid is to be understood as meaning a plant, plant part, seed or plant cell that carries said construct or said nucleic acid as a transgene due the result of an introduction of said construct or said nucleic acid by biotechnological means. The plant, plant part, seed or plant cell therefore comprises said expression cassette, said recombinant construct, or said recombinant nucleic acid.

Biosynthetic Pathway for Mogrosides

FIG. 1 shows the Mogroside biosynthetic pathway from Siraitia grosvenorii (Itkin et al., Proc Nat Acad Sci USA, 2016; 113:E7619-E7628; Seki et al., Bioscience, Biotechnology, and Biochemistry, 2018 VOL. 82, NO. 6, 927-934). Enzymes squalene epoxidases (SQEs), cucurbitadienol synthase (CDS), and epoxy hydrolases (EPHs) are involved in the successive steps of producing and converting mogrol precursors into mogrol. Mogrol precursors as intermediate product of the enzymatic pathway include but are not limited to: 2,3-oxidosqualene, 2,3;22.23-dioxidosqualene, 24,25-epoxycucurbitadienol, and 24,25-dihydroxycucurbitadienol. One of the characteristics of the mogroside biosynthesis pathway is that CDS, which is an oxidosqualene cyclase, uses 2,3;22,23-diepoxysqualene as its substrate to produce 24,25-epoxycucurbitadienol. Genomic analysis has revealed that S. grosvenorii harbors five genes that may encode squalene epoxidases (SQEs). Of these, two are strongly expressed during the initial stages of fruit development, as are CDS, Cytochromes P450 (CYP87D18), and epoxy hydrolases (EPHs), which catalyze the subsequent steps, and are predicted to be involved in the production of 2,3;22,23-diepoxysqualene. The S. grosvenorii genome contains eight genes encoding epoxide hydrolases which catalyze conversion of 24,25-epoxycucurbitadienol to 24,25-dihydroxycucurbitadienol. Importantly, enzymatic pathway for mogrosides according to the present disclosure is not limited by the mechanisms shown in FIG. 1a. Other terpene structures, mogrol precursors, enzyme-catalyzed reactions or conversion mechanisms are also possible. Certain enzyme may catalyze more than one type of reaction. For example, Cytochrome P450 enzymes catalyze conversion of cucurbitadienol to 11-hydroxy-cucurbitadienol or 11-oxo-cucurbitadienol, conversion of 24,25-epoxycucurbitadienol to 11-hydroxy-24,25 epoxycucurbitadienol. In some related embodiments, non-monk fruit Cucurbits can make tetracyclic triterpenoid compounds similar to mogrol, because at least one of the intermediates, such as triterpenes, exist in the cellular pathway. Moreover, given the related pathways for modifying tetracyclic triterpenoid require associated enzymes such as reductases, these related plants already express these associated network enzymes. In other related embodiments, an additional gene could be introduced into non-cucurbits or native enzymes with functioanalities that could be upregulated to allow for intermediate metabolite production or for the associated enzymes, in order to yield mogrol, mogrosides, and mogroside-based sweeteners.

After the formation of mogrol, a series of glycosylations occurs to add glucose molecules, at position C-3 and at position C-24, to produce Mogrosides I-VI with various degrees of glycosylation. The Roman numeral I, II, III, IV, V, and V respectively stand for the number of glucose unit(s) in the corresponding glycosylated mogroside, isomogroside, or oxomogroside. Two Uridine phosphorylase-dependent glycosyltransferases (UGTs) were shown to contribute to these steps. One of them is UGT720-269-1, which is strongly expressed in the initial stages of fruit development and transfers one glucose molecule each to the hydroxyl groups at positions C-24 and C-3 of mogrol to produce Mogroside IIE (C-3 and C-24 glucosylations) via mogroside I-A1 (C-24 glucosylation) as an intermediate. The second UGT is UGT94-289-3, which is strongly expressed in the latter stages of fruit development and adds sugars to the other sugars already present on the acceptor molecule. UGT94-289-3 was shown to add one glucose molecule each at positions C-2′ and C-6′ of the C-24 glucose of Mogroside ILE, which was added earlier by UGT720-269-1. UGT94-289-3 also adds a glucose molecule to position C-6′ of the glucose bound at position C-3 of Mogroside IIE, thereby sequentially catalyzing three or more sugar transfer reactions to produce mogrosides with five or more glucose units.

It is important to note that other natural plants by native genomes thereof may also express enzymes that produce mogrol precursors, but these plants do not naturally produce all enzymes in a coordinated fashion required to produce mogrosides. For instance, as shown in FIG. 1B, plants such as cucumber, melon, and watermelon naturally express cucurbitadienol synthases which are capable of producing cucurbitadienol, a common precursor to mogrol from Siratia or cucurbitacin from melons. However, other enzymes like the Cytochromes P450 enzymes, which are capable of altering the cucurbitadienol scaffold (Banerjee et al., Phytochem. Rev. 2018, 17:81-111) redirect this intermediate to other terpene derivatives. As these non-Siratia plants have other Cytochromes P450, hydrolases, epoxidases, and glycosylase genes, whose enzyme products may be promiscuous in the activities, and may not be expressed in a coordinated fashion for a mogroside pathway, such plants may have the enzymes for a mogroside pathway. Therefore, changes to the genomes of these non-Siratia plants, either by recombinant, gene editing, or other modern plant breeding technologies may allow for these non-Siratia plants to begin to produce mogrol and mogrosides.

As used herein, the term “mogroside pathway enzyme” encompasses any enzyme capable of catalyzing or facilitating biosynthetic reactions to produce mogrol precursor, mogrol, mogroside, and metabolites and/or derivatives thereof. Such mogroside pathway enzymes include but are not limited to the enzyme family of each of CDS, SQE, EPH, Cytocrome P450, and UGT.

Mogrol precursor broadly encompasses all possible terpene derivatives and intermediate products towards the production of mogrol product of the enzymatic pathway shown in FIGS. 1a and 1b, including but not limited to 2,3-oxidosqualene, 2,3;22.23-dioxidosqualene, 24,25-epoxycucurbitadienol, and 24,25-dihydroxycucurbitadienol, cucurbitadienol, 11-hydroxy-cucurbitadienol, 11-oxo-cucurbitadienol, etc. Mogrosides according to the present disclosure refer to any possible glycosylation products of mogrol, including but not limited to Siamenoside I, Siratose (a stereoisomer of Siamenoside I), Mogroside VI, Mogroside V, Isomogroside V, Mogroside IV, Mogroside III, Mogroside IIIE, Mogroside IIE, Mogroside IIA, Mogroside IE, Mogroside IA. Some of these structures are shown in FIG. 2. Other examples of mogrosides include but are not limited to Mogroside IIB, 7-Oxomogroside IIE, 11-Oxomogroside A1, Mogroside III A2, 11-Deoxymogroside III, 11-Oxomogroside IVA, 7-Oxomogroside V, and 11-Oxo-mogroside V. Metabolites and derivatives of mogrosides according to the present disclosure refer to any close variation of mogrosides through metabolic reaction, naturally occurring reaction, or non-naturally occurring reaction. Derivatives of mogrosides may comprise deletions, alterations, or additions of atom(s) or functional groups compared with standard mogrosides. However, metabolites and derivatives of mogrosides remain substantially the same function and characteristic of the standard mogrosides.

### BRIEF DESCRIPTION OF THE DRAWINGS

FIG. 1a shows the reported enzymatic pathway for production of mogrol and mogrosides in Siraitia grosvenorii. (Seki et al Bioscience, Biotechnology, and Biochemistry, 2018 VOL. 82, NO. 6, 927-934).

FIG. 1B shows enzymatic pathway for production of mogrol precursors in some natural plants. (Banerjee et al., Phytochem. Rev. 2018, 17:81-111).

FIG. 2 shows structures of mogrol and selected mogrosides derived thereof.

FIG. 3 shows the design of various expression cassettes comprising nucleotide sequences encoding mogroside pathway enzymes.

FIG. 4 shows the ultra-high performance liquid chromatography-time-of-flight mass spectrometry (UPLC-TOFMS) result of Mogroside standards.

FIG. 5 shows the UPLC-TOFMS (retention time) analytical result of Mogroside II detection in the leaves of the transgenic plants Nicotiana bentamiana respectively transformed with the expression cassettes pBing008 and pBing024, comparing with a control plant p019.

FIG. 6 shows the UPLC-TOFMS (retention time) analytical result of Mogroside II detection in the leaves of the transgenic Nicotiana bentamiana co-transformed with the expression cassettes pBing003 and pBing007, and the leaves of transgenic Nicotiana bentamiana co-transformed with the expression cassettes pBing006 and pBing015, comparing with a control plant p019.

FIG. 7 shows the UPLC-TOFMS (retention time) result of Mogroside II detection in the leaves of the transgenic Nicotiana bentamiana transformed with the expression cassette pBing008.

FIG. 8 shows the UPLC-TOFMS (MS spectra) result of Mogroside II detection in the leaves of the transgenic plant Nicotiana bentamiana transformed with the expression cassette pBing008.

FIG. 9 shows a photo image of dissection of a watermelon and various fruit parts thereof.

FIG. 10 shows the expression of the color-producing gene PSY1 in various fruit tissues according to Example 4.

FIG. 11 shows the expression of the 8 identified tissue specific genes of Table 5 in various tissues of Sugar Baby watermelon according to Example 4.

FIG. 12 shows the expression of the 8 identified tissue specific genes of Table 5 in various tissues of Charleston Gray watermelon according to Example 4.

FIG. 13 shows the analytical results of protein detection in various transgenic watermelon samples (transformed with pBing008).

FIG. 14 shows the chemiluminescence results of protein detection in the transgenic watermelon made by transformation with the expression cassette pBing008.

FIG. 15 shows the analytical results of protein detection in various tissues of transgenic watermelon sample 008SBE4-1, which was made by transformation with the expression cassette pBing008.

FIG. 16 shows the analytical results of protein detection in various tissues of transgenic watermelon sample 008SBE5-4, which was made by transformation with the expression cassette pBing008.

FIG. 17 shows the analytical results of protein detection in various tissues of transgenic watermelon sample 008CHE4-13, which was made by transformation with the expression cassette pBing008.

FIG. 18 shows the analytical results of protein detection in various tissues of transgenic watermelon sample 008CHE4-16, which was made by transformation with the expression cassette pBing008.

FIG. 19 shows the UPLC-TOFMS results of the transgenic watermelon comprising the expression cassette pBing008 in comparison with the control.

FIG. 20 shows the UPLC-TOFMS (MS spectra) results of the transgenic watermelon comprising the expression cassette pBing008.

FIG. 21a shows the UPLC-TOFMS results of a sample extract from fruits of the transgenic watermelon 008CH4-19.

FIG. 21b shows the UPLC-TOFMS results of a control sample to the fruit extract sample of the transgenic watermelon 008CH4-19, wherein the control sample is an extract of the wild type, unmodified fruit created and spiked with 100 ng/ml Mogroside IIE.

FIG. 22 shows the UPLC-TOFMS results of the seed coats from the transgenic watermelon samples 008SBE5-2 and 008CHE4-5, both comprising the expression cassette pBing008.

FIG. 23 shows the comparison of CDS gene expression levels in 31 transgenic watermelon leaves fruits. Expression levels of CDS were analyzed by RT-PCR and normalized to 10% of Actin expression (set as 1). Orange bars represent expression value from fruits whereas blue bars represent expression value from leaves.

FIG. 24 shows the comparison of CYP87 gene expression levels in 31 transgenic watermelon leaves fruits according to Example 6. Expression levels of CYP87 were analyzed by RT-PCR and normalized to 10% of Actin expression (set as 1). Orange bars represent expression value from fruits whereas blue bars represent expression value from leaves.

FIG. 25 shows the comparison of SQE gene expression levels in 31 transgenic watermelon leaves fruits according to Example 6. Expression levels of SQE were analyzed by RT-PCR and normalized to 10% of Actin expression (set as 1). Orange bars represent expression value from fruits whereas blue bars represent expression value from leaves.

FIG. 26 shows the comparison of EPH gene expression levels in 31 transgenic watermelon leaves fruits according to Example 6. Expression levels of EPH were analyzed by RT-PCR and normalized to 10% of Actin expression (set as 1). Orange bars represent expression value from fruits whereas blue bars represent expression value from leaves.

FIG. 27 shows the comparison of EPH gene expression levels in 31 transgenic watermelon leaves fruits according to Example 6. Expression levels of EPH were analyzed by RT-PCR and normalized to 10% of Actin expression (set as 1). Orange bars represent expression value from fruits whereas blue bars represent expression value from leaves.

FIG. 28 shows the analytical results of standard Mogroside IIE by UPLC-MS analysis. Shown on the left is the overlay of three chromatograms of three different concentrations of Mogroside IIE: 1000 pg/ml, 500 pg/ml and 250 pg/ml. Shown on the right is the signature ion peaks of Mogroside IIE standards after fragmentation.

FIG. 29 shows the analytical results of the detection of Mogroside IIE in the metabolite extracts of TO watermelon fruit sample 008CHE4-19, according to Example 6. Extracted ion chromatograms (m/z 423.36) of LC-MS are shown on the left. The ion intensities of mass spec fragments are shown on the right.

FIG. 30 shows results of CDS gene expression in samples of T1 transgenic watermelons according to Example 6. DNA from 32 transgenic plant samples were amplified using multiplex-PCR. The identities and genotyping conclusions of all samples were listed in the table on the left and right.

FIG. 31 shows the analytical results of the detection of Mogroside IIE in the metabolite extracts of T1 watermelon fruit sample 008DLE11-4-S4, according to Example 6. Extracted ion chromatograms (m/z 423.36) of LC-MS are shown on the left. The ion intensities of mass spectroscopic fragments are shown on the right.

FIG. 32 shows the analytical results of the detection of Mogroside IIE in the metabolite extracts of T1 watermelon fruit sample 008DLE11-2-S1, according to Example 6. Extracted ion chromatograms (m/z 423.36) of LC-MS are shown on the left. The ion intensities of mass spectroscopic fragments are shown on the right.

FIG. 33 shows the analytical results of the detection of Mogroside IIE in the metabolite extracts of T1 watermelon fruit sample 008DLE11-9-S3, according to Example 6. Extracted ion chromatograms (m/z 423.36) of LC-MS are shown on the left. The ion intensities of mass spectroscopic fragments are shown on the right.

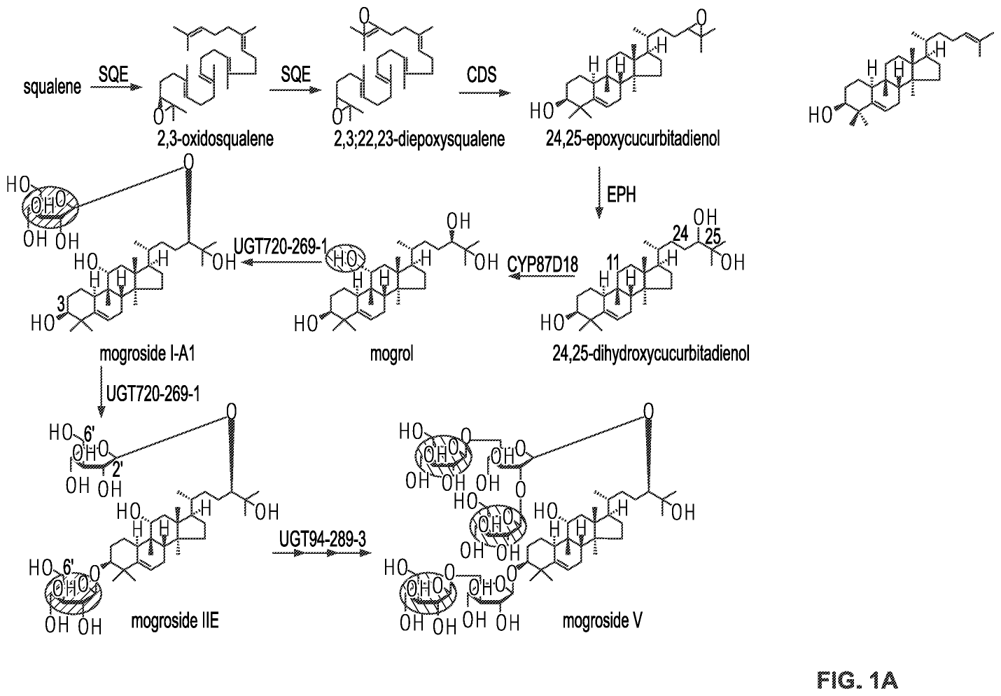

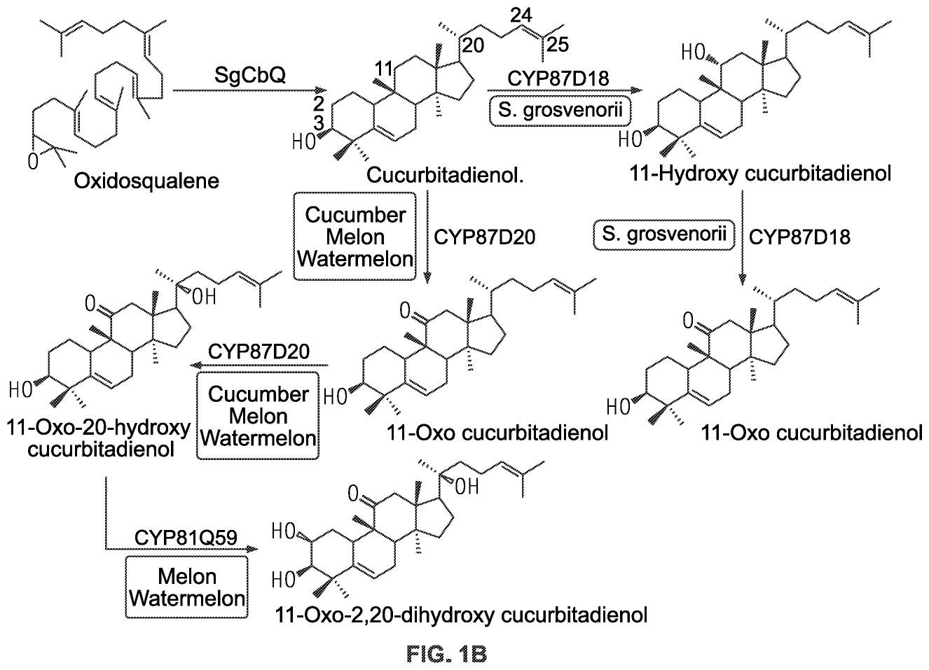

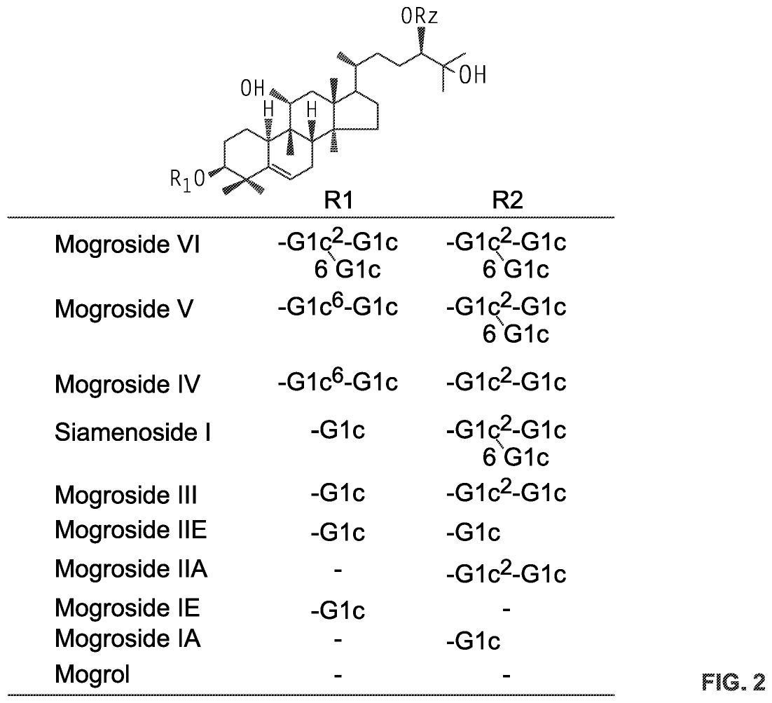

### DETAILED DESCRIPTION

The present document generally describes transgenic plants and biosynthetic systems thereof for making mogrol/mogroside pathway enzymes and mogrosides, and methods for making such transgenic plants. The following sections provide embodiments that describe the subject matter in detail.

Construction of Expression Cassettes and Vectors

In some embodiments, the present disclosure describes a transgenic plant comprising a genomic transformation event, wherein the genomic transformation event produces a non-native expression or concentration of mogroside pathway enzyme(s), wherein the transgenic plant biosynthetically produces non-native mogrol precursors, mogrol, mogrosides, and/or metabolites or derivatives thereof.

In other related embodiments the present disclosure describes a gene-edited plant comprising a genomic transformation event, wherein the genomic transformation event produces a non-native expression or concentration of mogroside pathway enzyme(s), wherein the transgenic plant biosynthetically produces non-native mogrol precursors, mogrol, mogrosides, and/or metabolites or derivatives thereof. In at least these embodiments, various genome editing tools, such as transcription activator-like effector nucleases (TALENs), zinc-finger nucleases (ZFNs), and meganucleases (MNs), can be used to obtain the desired plant with non-native mogrol precursors, mogrol, mogrosides, and/or metabolites or derivatives thereof.

As described further herein, the gene-edited plant may comprise SEQ ID NO: 1-31. In some example embodiments the gene-edited plant comprises an expression cassette, or transformation event, which includes one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 1-31.

In some embodiments, the present disclosure describes a transgenic plant comprising non-native mogrol precursors and/or mogrol, wherein the transgenic plant biosynthetically produces mogrol, mogrosides, and/or metabolites or derivatives thereof.

In some embodiments, the transgenic plant according to the present disclosure has a genomic transformation event, wherein the genomic transformation event comprises an expression cassette, wherein the expression cassette comprises one or more of the nucleotide sequences as set forth in SEQ ID NO: 1-31. In some embodiments, the expression cassette of the transgenic plant comprises one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 1-31.

The expression cassette herein has been designed and constructed via suitable recombinant gene techniques prior to plant transformation.

In some embodiments of the transgenic plant, the nucleotide sequences set forth in SEQ ID NO: 1; SEQ ID NO: 2; SEQ ID NO: 3; SEQ ID NO: 4; or SEQ ID NO: 5 in the expression cassette are capable of encoding at least one enzyme selected from the group consisting of CDS, Cytochromes P450, EPH, SQE, UGT, and combinations thereof.

In some embodiments, the expression cassette further comprises one or more components selected from the group consisting of promoter, nucleotide sequences of interest, epitope tag, terminator, spacer, and combinations thereof.

In certain embodiments, the expression cassette further comprises one or more promoters. In some embodiments, the one or more promoters is a strong promoter. In other embodiments, one or more promoters is a weak promoter. In yet other embodiments, the one or more promoters has one or more nucleotide sequences set forth in SEQ ID NO: 6-17. In yet other embodiments, the one or more promoters has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 6-17.

In certain embodiments, the expression cassette further comprises one or more epitope tags, wherein the one or more epitope tags has one or more nucleotide sequences set forth in SEQ ID NO: 18-22. In other embodiments, the one or more epitope tags has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 18-22.

In certain embodiments, the expression cassette further comprises one or more terminators, wherein the one or more terminators has one or more nucleotide sequences set forth in SEQ ID NO: 23-27. In other embodiments, the one or more terminators has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 23-27.

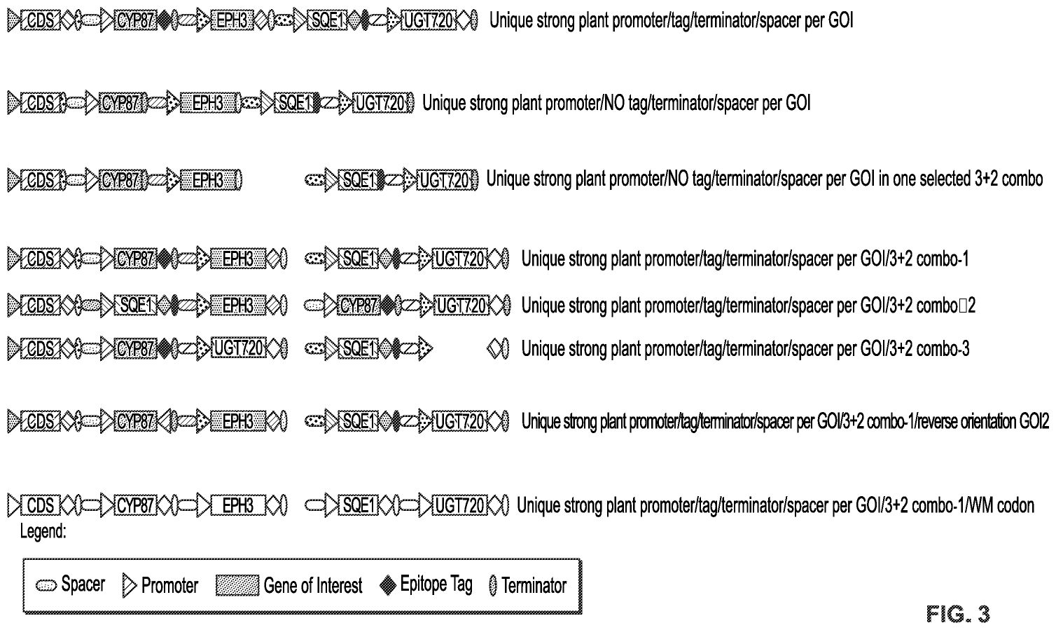

FIG. 3 shows some non-limiting example designs of the expression cassettes according to the present disclosure. Each of the genes of interest, for example, the nucleotide sequence encoding CDS, is operably linked to a promoter sequence and a nucleotide sequence encoding an epitope tag, wherein the epitope is operably linked to a terminator sequence, thereby forming an expressible gene as Promoter-CDS-Epitope tag-Terminator. Such “expressible genes” can be further modified by operably linked through space sequences (spacers) to alter expression or the enzyme product produced by the “expression cassettes.” In some embodiments, the expression cassette of the transgenic plant comprises one or more expressible genes and one or more spacers, wherein, each expressible gene comprises a gene sequence of interest selected from the group consisting of a nucleotide sequence encoding CDS, a nucleotide sequence encoding Cytochromes P450 (CYP87D18), a nucleotide sequence encoding EPH, a nucleotide sequence encoding SQE, a nucleotide sequence encoding UGT720, and combinations thereof.

In some embodiments, the expression cassette of the present disclosure further comprises one or more reporter gene sequences encoding and expressing one or more reporter proteins. The reporter proteins include but are not limited to kanamacin resistant protein (KAN), hygromycin resistant protein (Hyg), green fluorescent protein (GFP), and green fluorescent protein (RFP). In some embodiments, the one or more reporter genes has one or more nucleotide sequences set forth in SEQ ID NO: 28-31.

In other embodiments, the one or more reporter genes has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 28-31. In some embodiments, the nucleotide SEQ ID NO: 28 is capable of encoding KAN, the nucleotide SEQ ID NO: 29 is capable of encoding Hyg, the nucleotide SEQ ID NO: 30 is capable of encoding GFP, and the nucleotide SEQ ID NO: 31 is capable of encoding RFP. In certain embodiments, the expression cassette comprises at least one reporter gene selected from the group consisting of the nucleotide sequences as set forth in SEQ ID NO: 28-31. In other embodiments, the expression cassette of the transgenic plant comprises at least two reporter genes selected from the group consisting of the nucleotide sequences as set forth in SEQ ID NO: 28-31.

Table 1 shows various non-limiting examples representing the expression cassettes of the present application. Table 2 shows the components of the expression cassette pBing008, including five promoter sequences, five protein tags, five terminator sequences, and five transgenes of interest.

TABLE 1
expression cassettes encoding one or more mogroside pathway enzymes.

| | Transgenes encoding mogroside | | | | | | | | | | |
|---|---|---|---|---|---|---|---|---|---|---|---|
| | pathway enzymes | | | | | Reporter genes | | | | Promoter | Epitope |
| | SQE | CDS | CYP87 | EPH | UGT720 | GFP | RFP | Kan | Hyg | Type | Tag |
| pBing007 | X | | | | X | | X | | X | Weak | Yes |
| pBing003 | | X | X | X | | X | | | X | Weak | Yes |
| pBing008 | X | X | X | X | X | | | | X | Weak | Yes |
| pBing006 | X | | | | X | | X | | X | Strong | Yes |
| pBing015 | | X | X | X | | X | | | X | Strong | Yes |
| pBing024 | X | X | X | X | X | X | | | X | Strong | Yes |
| pBing010 | X | | | | X | | X | | X | Weak | No |
| pBing018 | | X | X | X | | X | | | X | Weak | No |
| pBing016 | X | X | X | X | X | X | | | X | Weak | No |
| pBing011 | X | | | | X | | X | | X | Strong | No |
| pBing002 | | X | X | X | | X | | | X | Strong | No |
| pBing028 | X | X | X | X | X | X | | | X | Strong | No |
| Ctr1 | | | | | | X | | | X | Strong | No |
| Ctr2 | | | | | | | X | | X | Strong | No |
| Ctr3 | | | | | | X | | X | | Strong | No |
| Ctr4 | | | | | | | X | X | | Strong | No |

As an example embodiment, the expression cassette pBing008 comprises promoter sequences, nucleotide sequences encoding mogroside pathway enzymes, nucleotide sequences encoding epitope tags, reporter genes encoding GFP and Hyg, and terminator sequences.

In pBing008, the nucleotide SEQ ID NO: 1 encodes CDS; the nucleotide SEQ ID NO: 2 encodes Cytochromes P450 (CYP87D18); the nucleotide SEQ ID NO: 3 encodes EPH; the nucleotide SEQ ID NO: 4 encodes SQE; the nucleotide in SEQ ID NO: 5 encode UGT720; the nucleotide in SEQ ID NO: 6 represents promoter TCTP; the nucleotide in SEQ ID NO: 7 represents promoter Fsgt-PFlt; the nucleotide in SEQ ID NO: 8 represents promoter CsVMV; the nucleotide in SEQ ID NO: 9 represents promoter HLV H12; the nucleotide in SEQ ID NO: 10 represents promoter PCLSV; the nucleotide in SEQ ID NO: 11 represents promoter MMV; the nucleotide in SEQ ID NO: 12 represents promoter CaMV e35S; the nucleotide in SEQ ID NO: 18 represents protein tag MYC; the nucleotide in SEQ ID NO: 19 represents protein tag HSV; the nucleotide in SEQ ID NO: 20 represents protein tag FLAG; the nucleotide in SEQ ID NO: 21 represents protein tag HA; the nucleotide in SEQ ID NO: 22 represents protein tag V5; the nucleotide in SEQ ID NO: 23 represents terminator CaMV 35S; the nucleotide in SEQ ID NO: 24 represents terminator UBQ3; the nucleotide in SEQ ID NO: 25 represents terminator HSP18.2; the nucleotide in SEQ ID NO: 26 represents terminator Pea3A; the nucleotide in SEQ ID NO: 27 represents terminator E9; the nucleotide in SEQ ID NO: 28 encodes reporter protein Hyg; the nucleotide in SEQ ID NO: 29 encodes reporter protein GFP.

In particular, the expression cassette pBing008 comprises the following seven expressible genes:
Promoter TCTP—the nucleotide sequence encoding CDS—the nucleotide sequence encoding the epitope tag MYC—the terminator CaMV 35S;
Promoter Fsgt-PFlt—the nucleotide sequence encoding Cytochromes P450 (CYP87D18)—the nucleotide sequence encoding the epitope tag HSV—the terminator UBQ3;
Promoter CsVMV—the nucleotide sequence encoding EPH3—the nucleotide sequence encoding the epitope tag FLAG—the terminator HSP18.2;
Promoter HLV H12—the nucleotide sequence encoding SQE—the nucleotide sequence encoding the epitope tag HA—the terminator Pea3A;
Promoter PCLSV—the nucleotide sequence encoding UGT720—the nucleotide sequence encoding the epitope tag V5—the terminator E9; Promoter MMV—the nucleotide sequence encoding GFP;
Promoter CaMV e35S—the nucleotide sequence encoding Hyg,
wherein the above seven expressible genes are operably linked by spacers to form the integrated expression cassette pBing008.

TABLE 2
Selected sequences of expression cassette pBing008.

| Promoter | | Transgene | | Protein Tag | | Terminator | |
|---|---|---|---|---|---|---|---|
| SEQ ID | TCTP | SEQ ID | CDS | SEQ ID | MYC = | SEQ ID | CaMV 35S |
| NO. 6 | (302 bp) | NO. 1 | | NO. 18 | EQKLI | NO. 23 | (180 bp) |
| | | | | | SEEDL | | |
| SEQ ID | Fsgt-PFlt | SEQ ID | CYP87D18 | SEQ ID | HSV = | SEQ ID | UBQ3 |
| NO. 7 | (585 bp) | NO. 2 | | NO. 19 | QPELA | NO. 24 | (405 bp) |
| | | | | | PEDPED | | |
| SEQ ID | CsVMV | SEQ ID | EPH3 | SEQ ID | FLAG = | SEQ ID | HSP18.2 |
| NO. 8 | (541 bp) | NO. 3 | | NO. 20 | DYKD | NO. 25 | (486 bp) |
| | | | | | DDDK | | |
| SEQ ID | HLV H12 | SEQ ID | SQE | SEQ ID | HA = | SEQ ID | Pea3A |
| NO. 9 | (500 bp) | NO. 4 | | NO. 21 | YPYDV | NO. 26 | (561 bp) |
| | | | | | PFYA | | |
| SEQ ID | PCLSV | SEQ ID | UGT720 | SEQ ID | V5 = | SEQ ID | E9 |
| NO. 10 | (542 bp) | NO. 5 | | NO. 22 | GKPIP | NO. 27 | (295 bp) |
| | | | | | NPLLG | | |
| | | | | | LDST | | |

In some embodiments, the expression cassette is carried on a plasmid so as to allow enzyme production by a host cell. In other embodiments, the expression cassette carried on a vector that allows for chromosomal integration, which allows enzymes to be expressed from a chromosome.

Construction of Plant Lines and Transformation

In some embodiments, the method of making the transgenic plants of the present disclosure is related to constructing plant lines and transforming the selected natural plants with the expression cassettes made according to the present disclosure.

It is generally known that the native expression of mogrol/mogroside pathway enzymes and natural production of native mogrol/mogrosides are only available in Siraitia grosvenorii. In some embodiments of the present application, the natural plants selected to be transformed with nucleotide sequences encoding mogrol/mogroside pathway enzymes are not Siraitia grosvenorii. In particular, the natural plants prior to transformation by their native genomes do not naturally produce all mogrol/mogroside pathway enzymes, and do not produce morgol and mogroside. In some embodiments, even the natural plants by the natural genomes thereof may produce one or more enzymes capable of producing mogrol precursors or mogrol, these plants do not produce non-native mogrosides naturally. In certain embodiments, the selected natural plants for transformation include wild-type, or untransformed, or non-transformed watermelons which do not by its native genome naturally produce detectable mogrol or mogroside.

Transformation of fast-growing economic fruits, vegetables, or plants that enable fast production of mogrosides are of more interest with respect to efficiency and cost. Non-limiting examples of fast growing plants are bush cherries, peaches and nectarines, apricot, radishes, plums and their relatives, sour (pie) cherries, apples, pears, sweet cherries, citrus, cucumbers, zucchinis, peas, turnips, and so on.

Transgenic plants according to the present disclosure are produced by combining a plant with a genomic transformation event thereby forming the transgenic plant, wherein the genomic transformation event produces a non-native expression or concentration of mogrol/mogroside pathway enzyme(s). In some embodiments, combining the plant with the genomic transformation event is performed using one or more of the following methods: use of liposomes, use of electroporation, use of chemicals that increase free DNA uptake, use of injection of the DNA directly into the plant, use of particle gun bombardment, use of transformation using viruses or pollen, use of microprojection, or use of Agrobacterium-mediated transformation. Preferably, the transgenic plants are made via Agrobacterium-mediated transformation method. In some embodiments, the Agrobacterium Tumefaciens was transformed with the expression cassette to create a transgenic agrobacterium, which was then used to transfect the plant of interest, and the successfully transformed plants were selected based on the expression of the reporter gene in the expression cassette.

In some embodiment, the transgenic plant is Nicotiana bentamiana, which was produced by transient transformation. First, Agrobacterium Tumefaciens Stain EHA105 was transformed with an expression cassette of the present application using a free-thaw method reported by Weigel et.al. (Transformation of agrobacterium using the freeze-thaw method, CSH Protoc. 2006 Dec. 1; 2006(7)). Briefly, chemically competent agrobacterium was prepared. After addition of the expression cassette, the mixture was alternately frozen in liquid nitrogen and thawed to liquid. The cells were then allowed to recover in a Lysogeny Broth (LB) medium and plated out on LB plates with a selected antibiotic.

Second, Nicotiana bentamiana plants was infected. Briefly, the transformed EHA105 agrobacterium was allowed to grow to generate a feasible population/culture. Selected Nicotiana bentamiana plants having appropriate maturity were chosen for transformation. An appropriate amount of transformed agrobacterium culture was loaded onto tissues of the Nicotiana bentamiana plants until an indication of completion. The plants loaded with the transformed agrobacterium culture were grown for an appropriate period of time before sampling and selection.

Third, the successfully transformed Nicotiana bentamiana plants were selected based on the leaves with expression of the reporter genes in the expression cassette.

In certain embodiments of transgenic Nicotiana bentamiana plants, the express cassette used to transform the Agrobacterium Tumefaciens Stain EHA105 and produce the transgenic Nicotiana bentamiana plants was pBing008. In other embodiments, the express cassette used were selected from those shown in Table 1. In some embodiments, the reporter gene of the expression cassette was GFP, and the selection of transformed Nicotiana bentamiana plants was based on the leaves thereof with expression of GFP.

In other embodiments, the transgenic plant is transgenic watermelon (Citrullus lanatus), which was produced by the following method. Briefly, first, Agrobacterium Tumefaciens Stain EHA105 was transformed with an expression cassette of the present application using the same free-thaw method. Second, watermelon seedlings with appropriate maturity were used for preparing explants for the transformation. Cotyledons were cut off from hypocotyls, collected and appropriately treated for transformation. Then, the transformed agrobacterium culture was added to these explants. After infection, explants were blotted on sterile paper towels and transferred to plates with a Murashige and Skoog (MS) medium. The plates were sealed and allowed for co-cultivation for an appropriate period of time. After co-cultivation, the explants were moved to growth chambers to allow for growing, under the selection of the threshold content of selected antibiotics.

In certain transgenic watermelon embodiments, the express cassette used to transform the Agrobacterium Tumefaciens Stain EHA105 and produce the transgenic watermelons was pBing008. In other embodiments, the express cassette used were selected from those shown in Table 1. In some embodiments, the reporter gene of the expression cassette was GFP, and the selection of transformed watermelon plants was based on the leaves thereof with expression of GFP.

In other embodiments, the plant was co-transformed by infection with two or more expression cassettes, wherein the express cassettes used were selected from those shown in Table 1.

Protein Expression in Transgenic Plants and Tissues Thereof

In some embodiments, the method of making the transgenic plants of the present disclosure is related to monitoring and analyzing the expression of mogrol/mogroside pathway proteins/enzymes by the expression cassette introduced in the transgenic plants.

In some embodiments, the tissues or parts of the transgenic plants made according to the present application were sampled and treated to obtain samples ready for analysis. The samples were further subject to analysis to detect the existence and/or content of proteins expressed by the gene of interests in the expression cassette.

In some embodiments, the leaves of the transgenic Nicotiana bentamiana plants made according to this disclosure were grounded in a protein extraction buffer and then were subject to centrifuge. The resultant supernatant was further diluted and then were used for antibody detection. The presence of each of the target proteins were confirmed by detection of chemiluminescent signals produced by binding of corresponding antibodies, as well as the size of the proteins, as indicated by the protein size ladder used as a control in each measurement. In some embodiments, the protein detection was performed by using the Jess instrument (Bio-Techne), which automates the protein separation and immunodetection of traditional Western blotting method for protein detection. In certain embodiments, a Signal/Noise ratio (S/N ratio)>3 was used as cutoff for positive signals for the purpose of analysis and selection.

In some embodiments, the transgenic plants showed existence of all five mogrol/mogroside pathway enzymes/proteins as follows: CDS, SQE, Cytochromes P450 (CYP87D18), UGT720, and EPH from the results of the protein detection. In certain embodiments, the transgenic plant is transgenic Nicotiana bentamiana. In other embodiments, the transgenic plant is transgenic watermelon.

Mogrol/Mogroside pathway enzymes were detected in various tissues of the transgenic plants of the present application, including but not limiting to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures, placenta, locule, mesocarp, rind, epidermis, or any other part or product of the transgenic plant. In certain embodiments, mogrol/mogroside pathway enzymes CDS, SQE, Cytochromes P450(CYP87D18), UGT720, and EPH were detected in placenta, locule, mesocarp, rind, and epidermis of the transgenic plant. In some embodiments, the expression of mogrol/mogroside pathway enzymes were tissue-specific. In certain embodiments, expression levels of CDS and UGT720 are lower than CYP87, SQE, and EPH. In other embodiments, the expression level EPH is significantly higher comparing with other mogrol/mogroside pathway enzymes particularly in fruit tissues.

Metabolic Modulation and Enzymatic Production of Mogrosides

In some embodiments, the method of making the transgenic plants of the present disclosure is related to analyzing the production of various non-native mogrosides. In certain embodiments, production of mogrosides by the transgenic plants is analyzed and compared to the corresponding control plants for the purpose of selection.

In some embodiments, tissues or parts of transgenic plants were extracted and/or purified to obtain samples ready for analysis. In some embodiments, UPLC coupled with TOFMS was used to analyze the metabolites in the tissues of transgenic plants. The existence of mogroside was determined by comparing the analytical result with the standard mogroside with respect to the retention time and the peak patterns of the MS spectra.

In some embodiments, the transgenic plants analyzed by UPLC-TOFMS showed signals of at least one mogroside, while the control plant showed no presence of mogroside from the analytical results.

In certain embodiments, the transgenic plants analyzed by UPLC-TOFMS showed at least one mogroside selected from the group consisting of Siamenoside I, Siratose, Mogroside VI, Mogroside V, Isomogroside V, Mogroside IV, Mogroside III, Mogroside IIIE, Mogroside II, Mogroside IIA, Mogroside IIA1, Mogroside IIA2, Mogroside IIE, MogrosideIIE2, Mogroside I, Mogroside IA, Mogroside IE, or any combinations thereof, while the control plant showed no presence of mogroside from the analytical results.

In other embodiments, the transgenic plants analyzed by UPLC-TOFMS showed at least one mogroside selected from the group consisting of Mogroside IA, Mogroside IE, Mogroside IIA, Mogroside IIA1, Mogroside IIA2, Mogroside IIE, Mogroside 11E2, or any combinations thereof, while the control plant showed no presence of mogroside from the analytical results.

Mogrosides were detected in various tissues of the transgenic plants of the present application, including but not limiting to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the transgenic plant. In certain embodiments of transgenic watermelon, mogrosides were detected in seed coat from fruit.

In some embodiments, the transgenic plant of the present disclosure is cultivatable and reproducible. A progeny or an ancestor of the transgenic plant is a source of non-native enzyme(s) enabling the progeny and the ancestor to produce mogrol, mogrosides, and/or metabolites or derivatives thereof. Propagation of the seed of the transgenic plant results in viable progeny thereof, wherein the progeny produces mogrol, mogrosides, and/or metabolites or derivatives thereof.

In some embodiments, the transgenic plant producing non-native mogrol/mogroside is a diploid plant, having diploid sets of chromosomes. In certain embodiments, the diploid transgenic plant produces seeds, wherein the seeds comprise non-native mogroside, and wherein propagation of the seeds of the diploid transgenic plant results in viable progeny thereof, wherein the progeny produces morgol, mogrosides, and/or metabolites or derivatives thereof. In some embodiments, the transgenic plant is a Cucurbitaceae/Curcubits. In some embodiments, the transgenic plant is a transgenic watermelon (Citrullus lanatus). In certain embodiments, the transgenic watermelon is diploid.

Mogroside-Containing Sweeteners and Consumables Derived from Transgenic Plants

In some embodiments, the present disclosure relates generally to a sweetener or sweetening composition comprising mogroside and/or metabolites or derivatives thereof, wherein the sweetener or sweetening composition is derived from a transgenic plant producing and comprising non-native mogrol/mogrosides. In certain embodiments, the sweetener or sweetening composition is derived from the mogrol/mogroside pathway transgenic plants made according to the present disclosure.

The mogrol/mogroside pathway transgenic plants of the present disclosure can derive mogroside-containing sweeteners upon appropriate processing. The resulting sweeteners could be used to provide low or non-caloric sweetness for many purposes. Examples of such uses to provide sweetness are in beverages, such as tea, coffee, fruit juice, and fruit beverages; foods, such as jams and jellies, peanut butter, pies, puddings, cereals, candies, ice creams, yogurts, bakery products; health care products, such as toothpastes, mouthwashes, cough drops, cough syrups; chewing gums; and sugar substitutes. In certain embodiments, the sweetener is in a juice of the transgenic plant according to the present application.

In some embodiments, the present disclosure also relates to methods of making the sweetener derived from transgenic plants producing non-native mogrol/mogrosides. The methods generally encompasses the steps including but not limited to pre-treatment cleaning and crushing of the transgenic plant or the parts thereof, extraction of the transgenic plant or the parts thereof, sedimentation and/or centrifuge, adsorption and/or separation, concentration and recovery to produce the crude sweetener, further purification, optional concentration/drying, and formulation. Means of extraction encompasses water-extraction at room temperatures, or heated temperature, or refrigerated temperature; extraction via organic solvent such as alcohol, et al. Means of separation and purification encompasses centrifuge, steeping, gravity sedimentation, filtration, micro-filtration, nano-filtration, ultra-filtration, reverse osmosis, chromatography, absorption chromatogram, exchanged resin purification, etc.

In certain embodiments, the sweetener is obtained from the leaves of the transgenic plant made according to the present disclosure. In other embodiments, the sweetener is obtained from the fruits of the transgenic plant made according to the present disclosure.

In some embodiments, the sweetener is obtained from transgenic watermelon according to the present disclosure, wherein the sweetener comprises non-native mogrosides produced by the transgenic watermelon.

While the forms of mogrol/mogroside pathway transgenic plants and methods of making the same described herein constitute preferred embodiments of this disclosure, it is to be understood that the disclosure is not limited to these precise forms. As will be apparent to those skilled in the art, the various embodiments described above can be combined to provide further embodiments. Aspects of the present transgenic plants, method, and process (including specific components thereof) can be modified, if necessary, to best employ the systems, methods, nodes and components and concepts of the present disclosure. These aspects are considered fully within the scope of the invention as claimed. For example, the various methods described above may omit some acts, include other acts, and/or execute acts in a different order than set out in the illustrated embodiments.

Further, in the transgenic plants and methods of making taught herein, the various acts may be performed in a different order than that illustrated and described. These and other changes can be made to the present systems, methods and articles in light of the above description. In general, in the following claims, the terms used should not be construed to limit the disclosure to the specific embodiments disclosed in the specification and the claims, but should be construed to include all possible embodiments along with the full scope of equivalents to which such claims are entitled. Accordingly, the invention is not limited by the disclosure, but instead its scope is to be determined entirely by the following claims.

All publications, patents and patent applications mentioned in the specification are indicative of the level of those skilled in the art to which this disclosure pertains.

The following examples illustrate preferred, but non-limiting embodiments of the present invention.

### EXAMPLES

### Example 1—Identification of Nucleotide Sequences Related to SEQ ID NOs: 1-31

Nucleotide sequences (full length cDNA, ESTs, or genomic) related to SEQ ID NOs: 1-31 are identified via previously reported non-patent literature of the mogroside pathway (Itkin et al., Proc Nat Acad Sci USA, 2016; 113:E7619-E7628) and by published patent applications WO2014086842 and WO2013076577.

### Example 2—Construction of Expression Cassette Comprising One or More Nucleotide Sequences Selected from SEQ ID NOs: 1-31

As shown in Table 1, various expression cassettes having different combinations of nucleotide sequences encoding the mogroside pathway enzymes were constructed. Construction of these expression cassettes was carried out following standard genetic engineering methods.

Briefly, expression cassettes were ordered from a gene synthesis vendor (GeneWiz) and assembled through enzymatic digestion and ligation. For example, pBing008 was assembled using five synthetic gene fragments: 1) TCTP promoter/CDS coding region/c-myc epitope tag/CaMV 35S terminator; 2) N3 spacer/FSgt-PFlt promoter/CYP87D18 coding region/HSV epitope tag/At UBQ3 terminator; 3) N5 spacer/CsVMV promoter/EPH3 coding region/FLAG epitope tag/At HSP18.2 terminator; 4) N8 spacer/HLV H12 promoter/SQE1 coding region/HA epitope tag/pea 3A terminator; 5) N7 spacer/PCSLV promoter/UGT720 coding region/5 epitope tag/E9 terminator. Expression cassettes 4 and 5 were assembled using unique Bsal restriction sites at their 5′ and 3′ ends into a pCAMBIA-based plant binary to create intermediate vector pBING003. Expression cassettes 1, 2, and 3 were assembled using unique Bsal restriction sites at their 5′ and 3′ ends into a pCAMBIA-based plant binary vector to create intermediate vector pBING005. A SbfI to SalI restriction fragment spanning expression cassettes 1, 2, 3 and the MMV promoter/eGFP gene of intermediate vector pBING005 was then subcloned into the SbfI to SalI restriction sites of intermediate vector pBING003 to create the final vector pBING008. This vector was verified by restriction digestion analysis using enzymes SphI+PstI and then confirmed by Sanger sequencing using a series of oligonucleotide primers that were designed to cover the entire T-DNA region of the binary vector.

### Example 3—Transgenic Plant Nicotiana bentamiana

Construction of expression cassettes: Various expression cassettes selected from Table 1 were constructed and used for transforming Nicotiana bentamiana and making the transgenic Nicotiana bentamiana plant. The expression cassette pBing008 comprises all five transgenes encoding mogroside pathway enzymes, two reporter genes respectively encoding GFP and Hyg, and nucleotide sequences respectively encoding an epitope tag, a weak promoter, and a terminator. Expression cassettes pBing003, pBing006, pBing007, pBing015, and pBing024 having different genetic combinations were constructed in the same way as described in Example 2.

Creation of transformed Agrobacterium: Agrobacterium Tumefaciens Stain EHA105 was transformed with one or more expression cassette plasmids (selected from Table 1) using a free-thaw method (Weigel, CSH Protoc. 2006 Dec. 1; 2006(7)). Briefly, chemically competent Agrobacterium was prepared. After addition of plasmids, the mixture was alternately frozen in liquid nitrogen and thawed to liquid in a 37 degree Celsius water bath. The cells were then allowed to recover in LB medium for about 1 hr and plated out on LB plates with Kanamycin.

Infection of the plant Nicotiana bentamiana: Briefly, the transformed EHA105 agrobacterium was grown overnight and then diluted until the OD600 reading reached 0.12. Nicotiana bentamiana plants that are six-week-old each having five leaves were chosen for transformation. The diluted transformed agrobacterium culture was loaded to a 5 mL syringe without needle, and about 1.5 mL was injected to the back side of the leaves until the leaves turned dark green. The plants loaded with the transformed agrobacterium culture were grown for another 10 days before sampling and selection. Four example transgenic Nicotiana bentamiana plants were respectively prepared by transformation with the following expression cassette(s):
pBing008 (5 genes, weak promoters)
pBing024 (5 genes, strong promoters)
pBing003+pBing007 (3+2 genes, weak promoters)
pBing006+pBing015 (3+2 genes, strong promoters)

Protein expression in tissue: About 50 mg of leaves were sampled into 1.7 mL microcentrifuge tubes, in which 500 μl protein extraction buffer (1×RIPA lysis buffer) were added. Leaf tissues were grounded in the extraction buffer before centrifugation to remove the debris. The supernatant was further diluted 3 times by extraction buffer before 4.5 μl of extract were used for antibody detection using the Jess instrument (Bio-Techne), which automates the protein separation and immunodetection of traditional Western blotting method for protein detection. Anti-rabbit antibodies for MYC, HSV, FLAG, HA and V5 tags were purchased from Thermo Fisher and diluted 50 times for use in Jess based western detection, which was performed using manufacturer's manual. The presence of each of the target proteins was confirmed by detection of chemiluminescent signals produced by binding of corresponding antibodies, as well as the size of the proteins, as indicated by the protein size ladder used as a control in each measurement.

As shown in Table 3, when Nicotiana bentamiana leaves were transformed with the expression cassette pBing008, all five target proteins can be detected in 10 days with a S/N ratio >3 used as cutoff for positive signals.

TABLE 3
Analytical results of mogroside pathway enzyme expression in the leaves of transgenic
Nicotiana bentamiana transformed with the expression cassette pBing008.

| | Expected | | | | | | |
|---|---|---|---|---|---|---|---|
| | MW | MW | | | | | |
| Enzyme | (kDa) | (kDa) | Height | Area | Width | S/N | Baseline |
| CDS-MYC | 87 | 82 | 5655.4 | 155357 | 25.8 | 11.3 | 4539.5 |
| SQE-HA | 59 | 60 | 7892.5 | 396755 | 47.2 | 15.2 | 3329.2 |
| CYP87-HSV | 55 | 54 | 44696.9 | 1346236 | 28.3 | 98.6 | 3009.1 |
| UGT720-V5 | 54 | 56 | 280524.4 | 6459802 | 21.6 | 566.9 | 6565.2 |
| EPH3-FLAG | 37 | 38 | 875910.3 | 22029626 | 23.6 | 1383.2 | 7918.6 |
| Actin | 42 | 48 | 14533 | 252483 | 16.3 | 49.1 | 4101.1 |

Metabolic modulation: About 100 mg of plant tissue were extracted in 500 μl extraction buffer (80% Methanol). After centrifuge, the supernatant was forced to pass through 0.2211M filter in order to remove remaining particles. Waters Acquity UPLC coupled by Waters Xevo Quadrupole Time of Flight Tandem Mass Spectrometer was used for metabolite analysis. For UPLC separation, Waters Acquity BEH C18 1.7 μm, 2.1×50 mm column was used with Water and Acetonitrile as solvents (both with 1% formic acid). For each analysis, 1.5 μl of sample was injected. MS/MS under negative ESI was used for detection of mogroside compounds. The collision energy was set to 30 V for detection of Mogroside IIs.

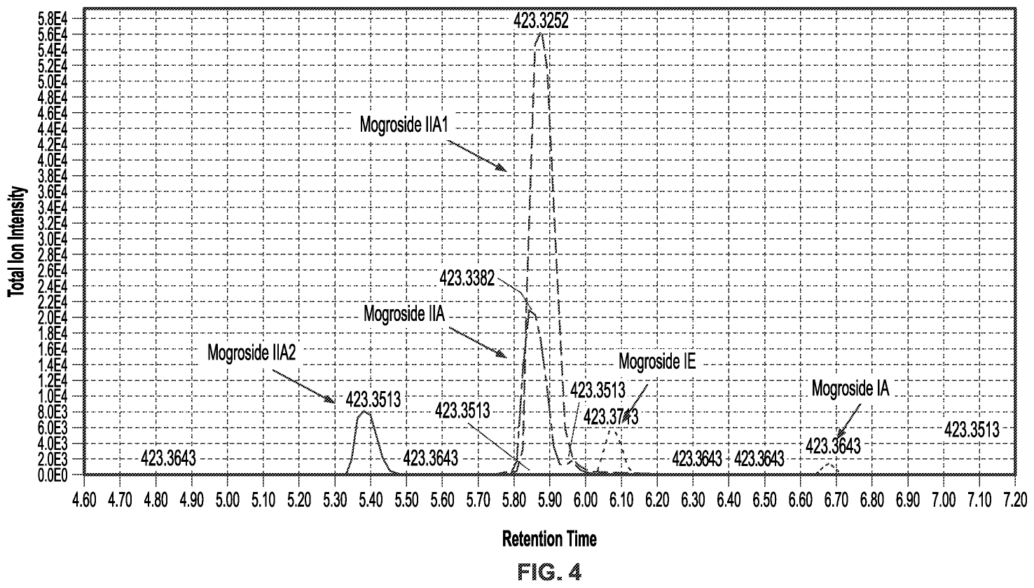

FIG. 4 shows the UPLA analytical results of standard Mogrosides. As can be seen, Mogroside IIA1 and Mogroside IIA are co-eluted at about 5.9 min retention time. Results also showed m/z 423 (signature peak for mogroside related compounds) across UPLC separation gradient. Mogroside standards were shown at very high concentration (0.1 mg/ml).

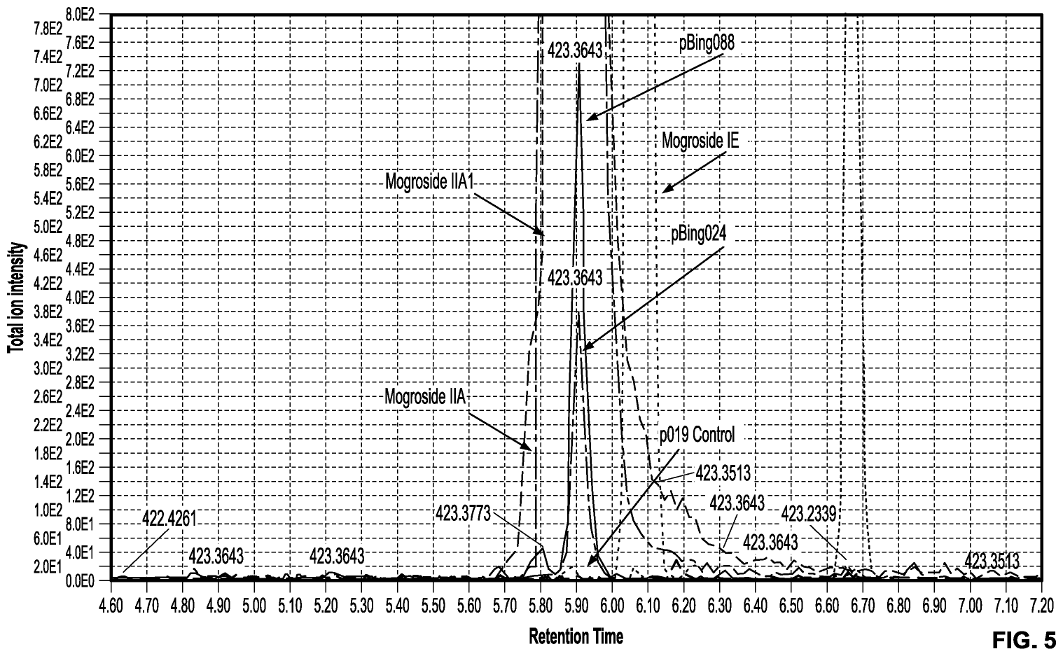

FIG. 5 shows the UPLC-TOFMS (retention time) analytical results of Mogroside II detection in the leaves of the transgenic plants Nicotiana bentamiana respectively transformed with the expression cassettes pBing008 and pBing024. As can be seen, comparing with the control plant p019 producing no mogroside peaks, both the pBing008 transgenic Nicotiana bentamiana and the pBing024 transgenic Nicotiana bentamiana produced Mogrosides II peaks, which is confirmed by the overlapped retention time with the mogroside standards.

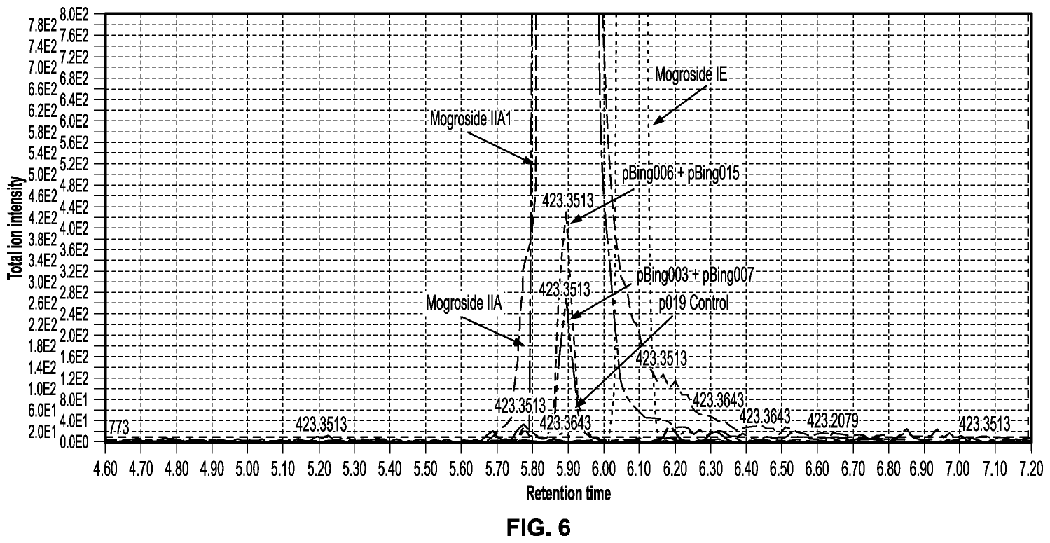

FIG. 6 shows the UPLC-TOFMS (retention time) analytical result of Mogroside II detection in the leaves of the transgenic Nicotiana bentamiana co-transformed with the expression cassettes pBing003 and pBing007, and the leaves of transgenic Nicotiana bentamiana co-transformed with the expression cassettes pBing006 and pBing015. As can be seen, comparing with the control plant p019 producing no mogroside peaks, both the pBing003+pBing007 transgenic Nicotiana bentamiana and the pBing006 and pBing015 transgenic Nicotiana bentamiana produced mogrosides II peaks, which is confirmed by the overlapped retention time of the mogroside standards.

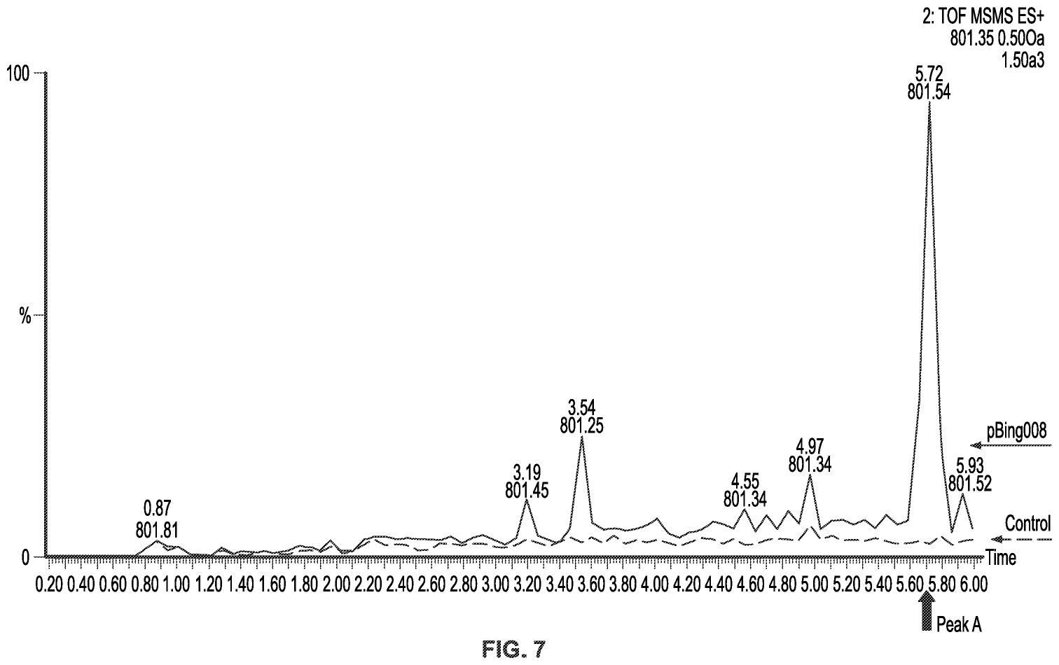

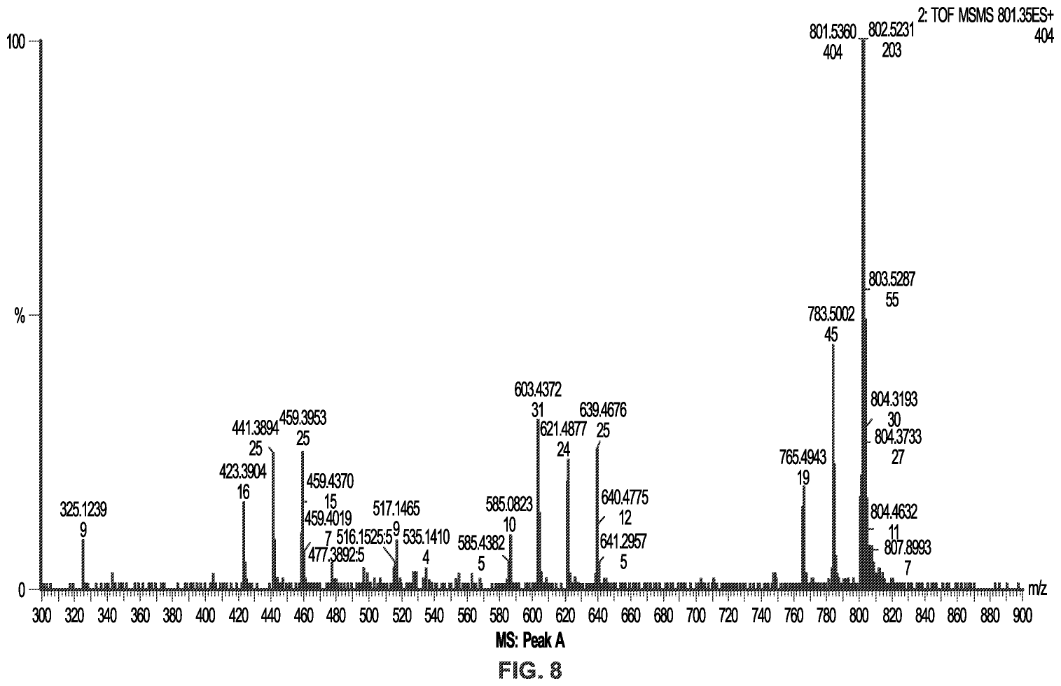

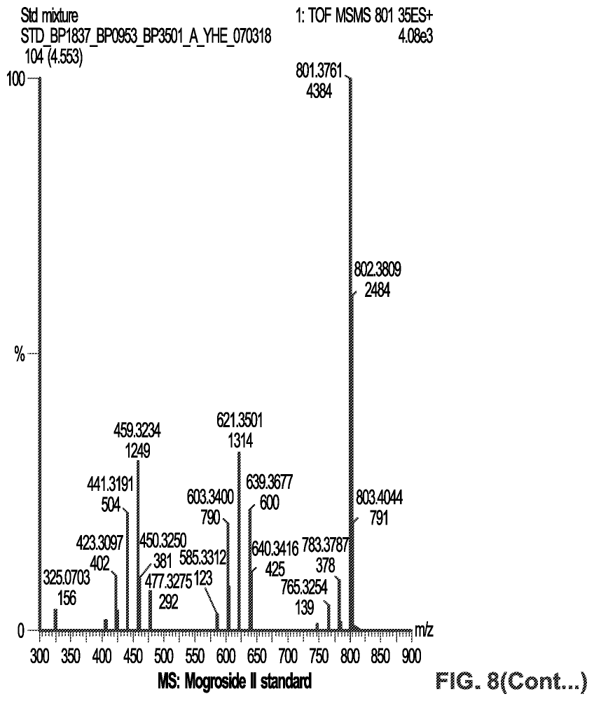

FIGS. 7 and 8 show the UPLC-TOFMS results of Mogroside IIA detection in the leaves of the transgenic Nicotiana bentamiana transformed with the expression cassette pBing008. As can be seen, Nicotiana bentamiana leaves infected with the expression cassette pBing008 produced Peak A, which shares the same characteristics of Mogroside IIA with respect to retention time (FIG. 6) and mass spectrum pattern (FIG. 7), indicating that the transgenic plant Nicotiana bentamiana transformed with the expression cassette pBing008 produces and comprises Mogroside II.

### Example 4: Assembled Watermelon Tissue Specific Transcriptomes

Watermelon fruit has great potential for production of non-caloric sweeteners due to its large size and popular flavor. To design a genome editing or cis-genic strategy for pathway engineering, it is critical to identify watermelon fruit specific promoters that enables optimal expression of genetic payloads. Identification of these promoters requires a high-resolution transcriptomic dataset, from which a list of genes that are specifically expressed in the edible portion of watermelon fruit can be generated. As of today, there is no publicly available transcriptomic resource that can distinguish different parts of watermelon fruits at different developmental stage.

The present study provided a bioanalytical approach for the detection and quantification of expression level of endogenous genes in various fruit parts of two commercial varieties of watermelon, Charleston Gray and Sugar Baby. The watermelons were grown, and tissues and developmental-stage specific samples thereof were collected. High quality RNAs from all these samples were extracted and more than 20 million RNA-seq reads were generated for each sample. The sequencing results and the normalized RNA-expression levels for each of the target gene in each sample were analyzed and quantified. A preliminary list of genes that are found to be highly enriched in the flesh of watermelon fruits were produced.

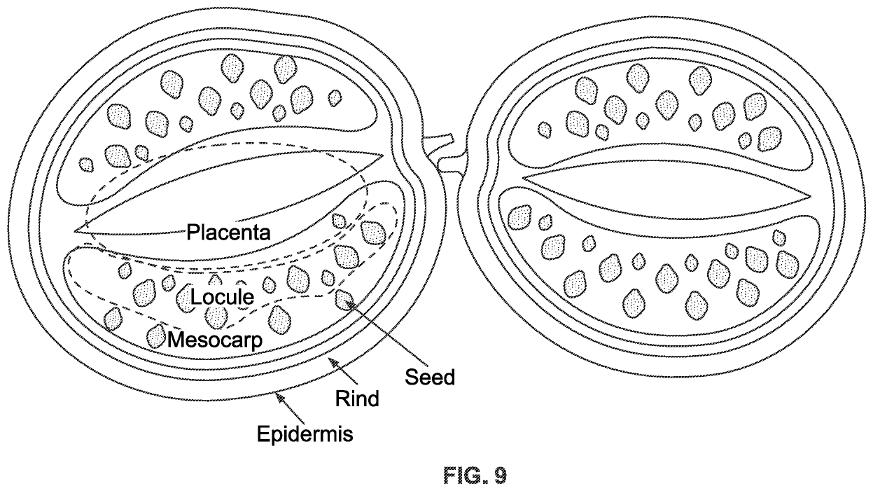

Table 4 summarizes various watermelon tissues samples that were collected for RNA-seq analysis. For each of the Sugar Baby species and the Charleston Gray species, 45 tissue samples of the fruit (5 types×3 ages×3 replicates=45), 6 samples of the leaf (2 ages×3 replicates=6), and 3 samples of the root were collected. FIG. 9 shows the dissection of watermelon and various parts thereof.

TABLE 4
Various watermelon tissues collected for RNA-seq analysis.

| Variation | Variable 1 | Variable 2 | Variable 3 | Variable 4 | Variable 5 |
|---|---|---|---|---|---|
| Species | Sugar | Charleston | | | |
| Variety | Baby | Gray | | | |
| Tissues | Leaf | Root | Fruit | | |
| Leaf Ages | Young | Mature | | | |
| Fruit Ages | 10 days | 26 days | 42 days | | |
| Fruit | Epidermis | Rind | Mesocarp | Locule | Placenta |
| Dissection | | | | | |

All RNA was checked by NanoDrop and BioAnalyzer. Total RNA amount per sample is about 1 microgram (μg) or more. Purity was set as OD260/280=1.8-2.2 and OD260/230≥2.0. The integrity will of RNA was checked by RIN numbers by Bioanalyzer to be >7. As a result of the RNA-seq analysis, minimal reads obtained from all samples are 20.8 million, and the average number of reads is 369 million for Charleston Gray and 37.8 million for Sugar Baby, respectively.

For the RNA-seq data analysis, low quality reads (q=30) were filtered out. The clean reads were aligned to the Charleston Gray reference genome (Wu et al., Genome of ‘Charleston Gray’, the principal American watermelon cultivar, and genetic characterization of 1,365 accessions in the U.S. National Plant Germplasm System watermelon collection. Plant Biotechnology Journal, 2019). The resulting alignment rates varied between 89.7 and 93.58. Gene counts for each sample were calculate. Samples were then normalized to account for differences in library depths.

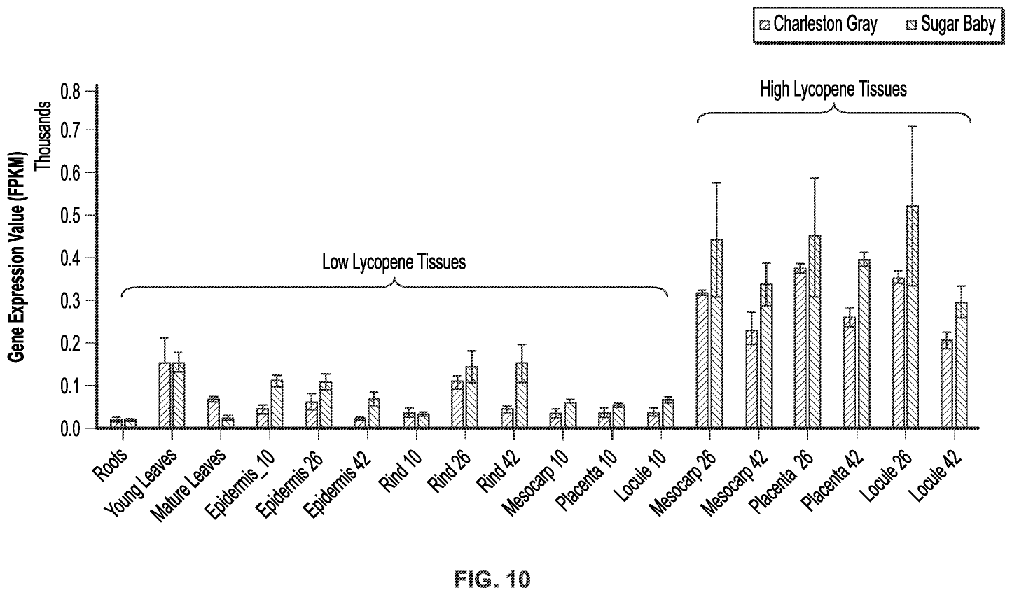

One hallmark of a developed watermelon fruit from both Charleston Gray and Sugar Baby is the pink/red flesh. Lycopene and β-Carotene are responsible for the fruit and it is known that the production of these pigments is control by the phytoene synthase gene PSY1 (Wang et al., Developmental Changes in Gene Expression Drive Accumulation of Lycopene and β-Carotene in Watermelon, Journal of the American Society for Horticultural Science, 2016, 141(5), 434-443). From this RNA-seq dataset, the expression of PSY1 gene is highly correlated with the accumulation of the pink/red color: the highest expression levels are detected in mesocarp, placenta, and locule tissues in fruits 26 days and 42 days old, which are the exact tissues that show visible pink and red colors as shown in FIG. 10. The correlation of PSY1 gene expression and tissue color validates that the RNA-seq dataset produced by this project is biologically meaningful.

As the next step, expression of all 22545 genes from 36 groups of samples were screened to identify additional fruit specific genes, with similar expression pattern to PSY1. The criteria are defined as: (1) Gene expression is highly enriched in 26-day-old and 42-day-old fruits, in the mesocarp, placenta and locule tissues (referred to as “target tissues”). The expression levels should be more than 5×higher than the expression in the rind, and more than 20×higher than in the root, leave and epidermis of fruits; (2) The expression level in target tissues should be >100 FPKM (Fragments Per Kilobase of transcript per Million mapped reads), to eliminate low abundance, yet tissue specific genes; and (3) The expression characters should meet both criteria in both varieties.

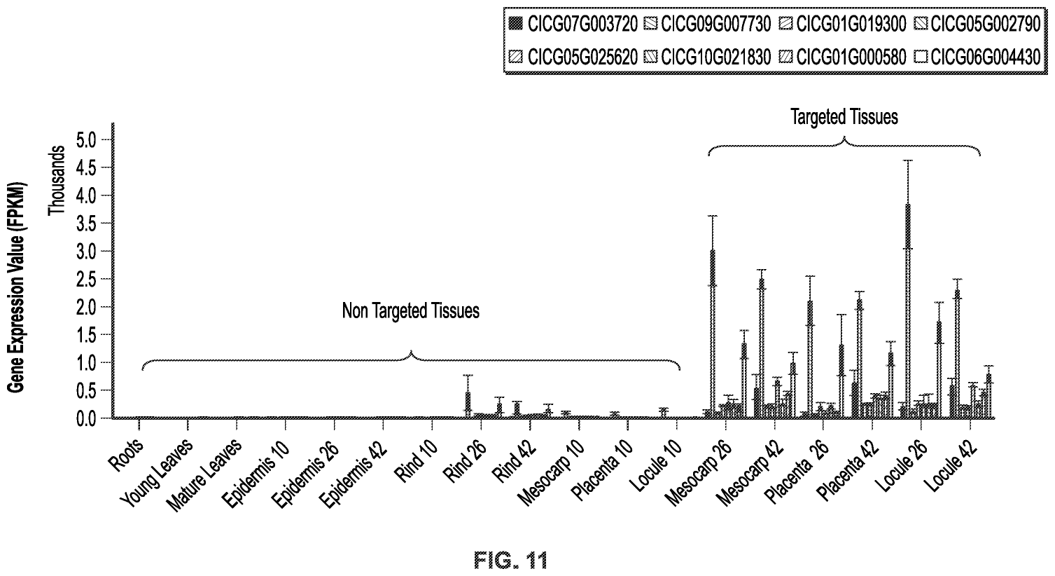

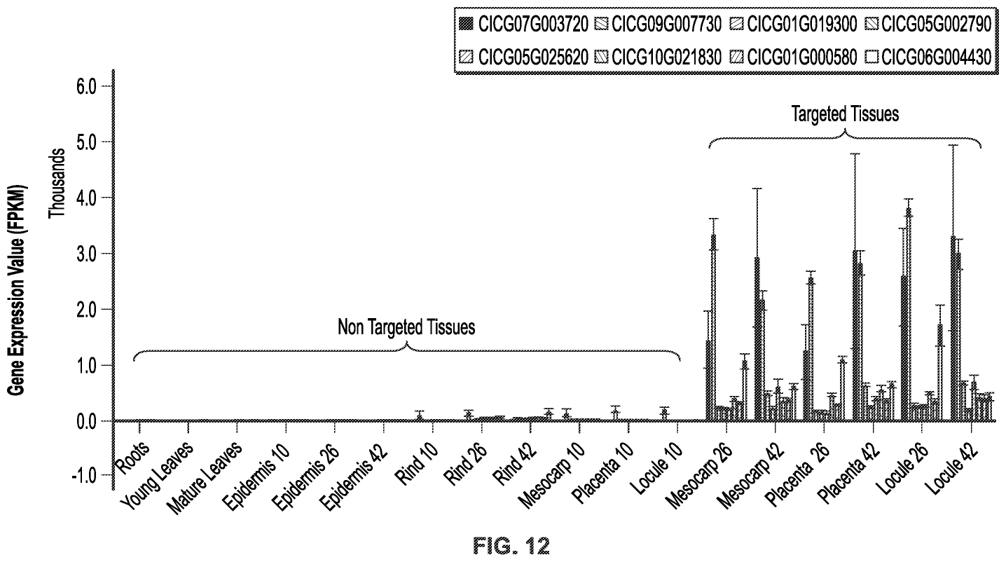

As a result, 8 genes were identified as such target tissue specific genes (using Charleston Gray Reference Genome Identifier http://cucurbitgenomics.org/). Interestingly, most of these genes are predicted to be involved in plant metabolism, which are indeed expected to be enriched during the fruit maturation stage, as shown in Table 5. Their expression enrichment of the genes in various tissues parts of the watermelon samples according to Table 4 is visualized in FIGS. 11-12. The gene expression dataset from this study could be a unique resource for discovery of tissue specific genes and promoters for the design and production of transgenic plants as well as analysis and quantification of expression of the target genes in the transgenic plants.

TABLE 5
The identified tissue specific genes of watermelon from the RNA-seq analysis.

| Gene ID | Functional Annotation |
|---|---|
| ClCG07G003720 | Subtilisin-like serine protease |
| ClCG09G007730 | 1-aminocyclopropane-1-carboxylate oxidase, putative |
| ClCG01G019300 | UDP-glycosyltransferase 1 |
| ClCG05G002790 | Sterol desaturase, putative |
| ClCG05G025620 | Lipase |
| ClCG10G021830 | Vesicular glutamate transporter 2.1 |
| ClCG01G000580 | Bidirectional sugar transporter N3 |
| ClCG06G004430 | Desiccation-related protein PCC13-62 |

### Example 5-Transgenic Watermelon

Construction of expression cassettes: Like the making of transgenic Nicotiana bentamiana in Example 3, various expression cassettes were prepared according to Table 1.

Creation of transformed Agrobacterium: Same procedure provided in Example 3 was followed to create transformed Agrobacterium.

Infection of watermelon: Two commercial varieties of watermelon, Charleston Gray and Sugar Baby, were used as hosts. Five-day-old watermelon seedlings were used for preparing explants for the transformation. Cotyledons were cut off from hypocotyls and collected in petri plates filled with sterile water. Two attached cotyledons were split by cutting through remaining hypocotyl segment and cotyledonary explants were cut into 2 mm pieces ready for transformation. For transformation, Agrobacterium culture was added to these explants and vacuumed for 5 minutes. After infection, explants were blotted on sterile paper towels and transferred to filter disks in petri plates with MS medium. The plates were sealed and placed at 25° C. for 3 days in the dark for co-cultivation.

Examples of transgenic watermelon were prepared by transformation with the following expression cassette(s):
pBing008 (5 genes, weak promoters)
pBing028 (5 genes, strong promoters, Hgy and GFP reporter proteins)

TABLE 6
Component sequences of expression cassette pBing028.

| Promoter | | Transgene | | Protein Tag | | Terminator | |
|---|---|---|---|---|---|---|---|
| SEQ ID | TCTP (302 | SEQ ID | CDS | SEQ ID | MYC = | SEQ ID | CaMV 35S |
| NO. 13 | bp) | NO. 1 | | NO. 18 | EQKLI | NO. 23 | (180 bp) |
| | | | | | SEEDL | | |
| SEQ ID | Fsgt-PFlt | SEQ ID | CYP87D18 | SEQ ID | HSV = | SEQ ID | UBQ3 |
| NO. 14 | (585 bp) | NO. 2 | | NO. 19 | QPELA | NO. 24 | (405 bp) |
| | | | | | PEDPED | | |
| SEQ ID | CsVMV | SEQ ID | EPH3 | SEQ ID | FLAG = | SEQ ID | HSP18.2 |
| NO. 15 | (541 bp) | NO. 3 | | NO. 20 | DYKD | NO. 25 | (486 bp) |
| | | | | | DDDK | | |
| SEQ ID | HLV H12 | SEQ ID | SQE | SEQ ID | HA = | SEQ ID | Pea3A |
| NO. 16 | (500 bp) | NO. 4 | | NO. 21 | YPYDV | NO. 26 | (561 bp) |
| | | | | | PFYA | | |
| SEQ ID | PCLSV | SEQ ID | UGT720 | SEQ ID | V5 = | SEQ ID | E9 (295 |
| NO. 17 | (542 bp) | NO. 5 | | NO. 22 | GKPIP | NO. 27 | bp) |
| | | | | | NPLLG | | |
| | | | | | LDST | | |

Protein expression in transgenic watermelon: Same procedure provided in Example 3 was followed to monitor and analyze protein expression in transgenic watermelon samples.

Table 7 shows the results of ploidy, metabolites, and gene expression of transgenic watermelon samples. Expression levels were quantified using Q-RT-PCR from leaf RNA samples. The expression levels were normalized to the predefined criteria (set as 1). For metabolite results, *** means abundant; ** means clear presence; * means likely presence. As shown in Table 7, when watermelon leaves were transformed with the expression cassette pBing008, or pBing028, all five target mogroside pathway proteins can be detected in the corresponding transgenic watermelon plants. In certain transgenic watermelon samples, the leaves are shown to have clear presence or abundant Mogroside IIE. It is important to note that, transgenic watermelon 008CHE4-19 having diploid chromosomes produced seeds and fruits, wherein the leaves of 008CHE4-19 had a likely presence of Mogroside IIE and abundant Mogroside IIE shown in metabolite results. Comparatively, transgenic watermelons having polyploidy such as triploid (3×) or tetraploid (4×) only showed flowering but did not ultimately produce seeds or fruits, or did not produce mogrosides in leaves or other tissues. These results surprisingly indicate that the ability of a transgenic watermelon to produce fruits and seeds was unexpected, and that the chromosomal ploidy may be an important factor to the reproducibility of transgenic watermelon producing non-native mogrosides.

TABLE 7
Ploidy, metabolites, and gene expression of transgenic watermelon samples.

| | | CDS | CYP87 | EPH3 | SQE | UGT720 | | | MIIE |
|---|---|---|---|---|---|---|---|---|---|
| | Plant ID | Expression | Expression | Expression | Expression | Expression | Ploidy | Status | (Leaf) |
| 1 | 008CHE1-1 | 1.0 | 166.1 | 7.1 | 83.7 | 2.2 | 4X | Flowering | |
| 2 | 008CHE1-5 | 1.1 | 224.1 | 6.0 | 127.6 | 1.3 | 4X | Flowering | ** |
| 3 | 008CHE4-19 | 0.9 | 35.5 | 5.6 | 459.9 | 7.8 | 2X | Fruit | * |
| 4 | 008CHE6-2 | 1.7 | 279.8 | 8.5 | 144.5 | 3.5 | 4X | Flowering | * |
| 5 | 008DLE10-1 | 1.3 | 253.9 | 1.4 | 256.6 | 4.0 | 3X | Flowering | |
| 6 | 008DLE10-2 | 2.3 | 413.2 | 49.1 | 154.4 | 7.9 | 4X | Flowering | |
| 7 | 008DLE12-1 | 2.3 | 35.4 | 12.7 | 404.9 | 21.5 | 4X | Flowering | |
| 8 | 008DLE12-14 | 1.4 | 77.2 | 82.0 | 446.4 | 11.0 | 3X | Flowering | * |
| 9 | 008DLE12-3 | 1.9 | 469.6 | 35.1 | 232.3 | 4.4 | | Flowering | ** |
| 10 | 008DLE12-8 | 5.3 | 1247.3 | 62.5 | 467.2 | 26.6 | 4X | Flowering | |
| 11 | 008SBE2-2 | 1.1 | 546.9 | 30.0 | 170.5 | 6.4 | 4X | Flowering | |
| 12 | 028DLE10-1 | 2.8 | 336.0 | 27.5 | 114.9 | 2.9 | 4X | Flowering | * |
| 13 | 028DLE15-1 | 7.8 | 453.0 | 94.8 | 174.8 | 10.8 | 4X | Flowering | * |
| 14 | 028DLE20-1 | 2.7 | 401.6 | 126.1 | 341.2 | 11.8 | 4X | Flowering | * |
| 15 | 036CHE62 | 3.9 | 3.2 | 44.4 | 242.2 | 20.4 | 3X | Flowering | |
| 16 | 037CHE23 | 6.2 | 305.6 | 56.6 | 374.9 | 28.9 | 4X | Flowering | |
| 17 | 037CHE24 | 3.4 | 5.3 | 4.8 | 291.4 | 6.1 | 3X | Fruit | |

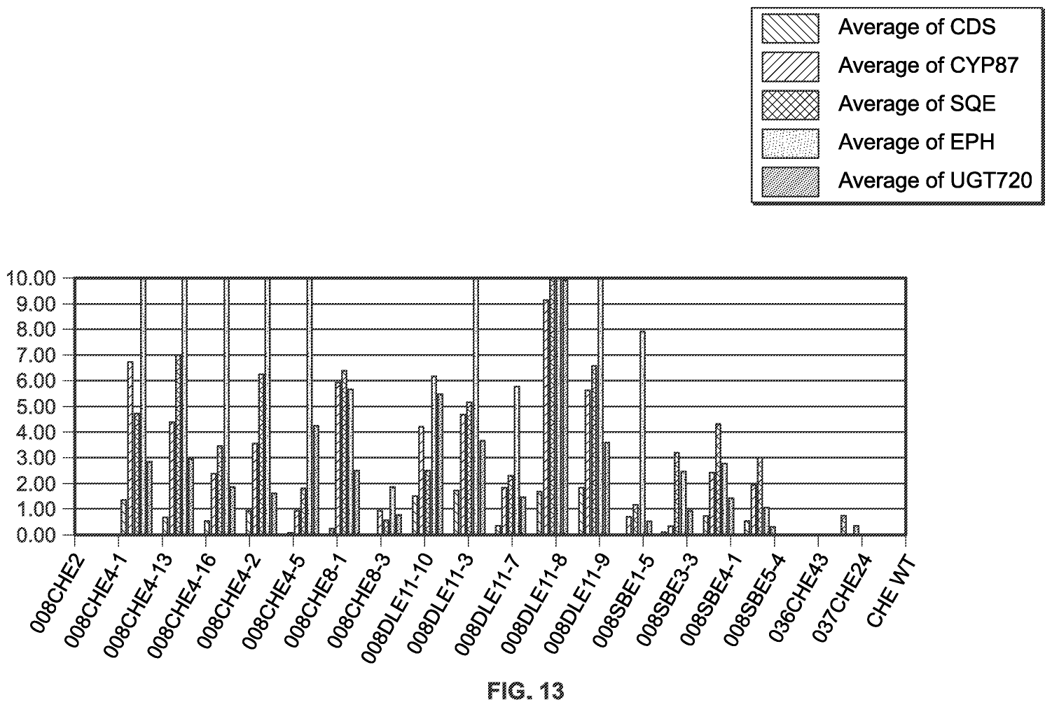

FIG. 13 shows the analytical results of protein detection various transgenic watermelon samples (transformed with pBing008). On Y-axis, 10% of Action expression was set as value 1.0. Expression of EPH is higher than the range and not shown in this graph. As can be seen, all transgenic watermelon samples showed high expression of all five mogroside pathway transgenes. Sample 008DLE11 clusters showed high expression of all transgenes. Sample 008DLE11-8 fruit has the highest expression and also produced 50 seeds.

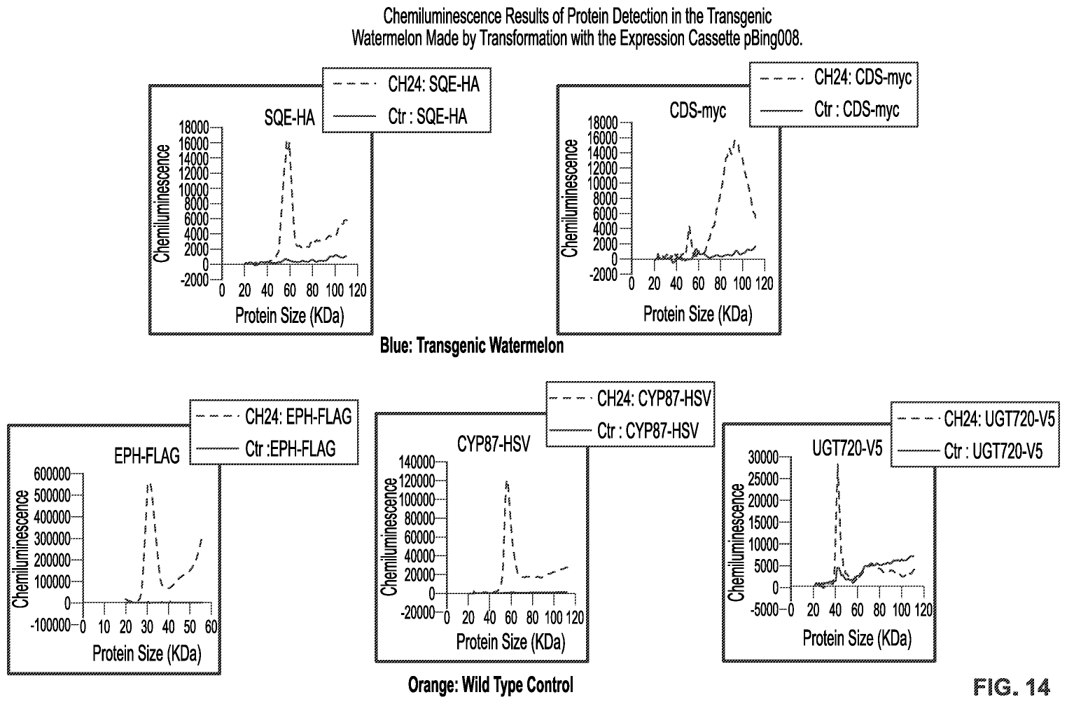

FIG. 14 shows chemiluminescence results of protein detection in the transgenic watermelon made by transformation with the expression cassette pBing008. As can be seen, expression of all five target mogroside pathway enzymes were found in the transgenic watermelon.

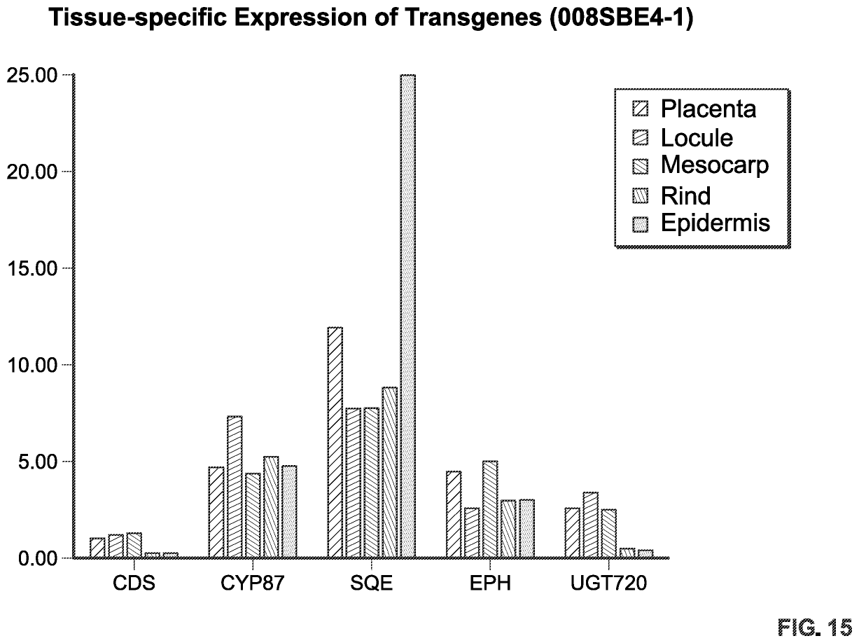

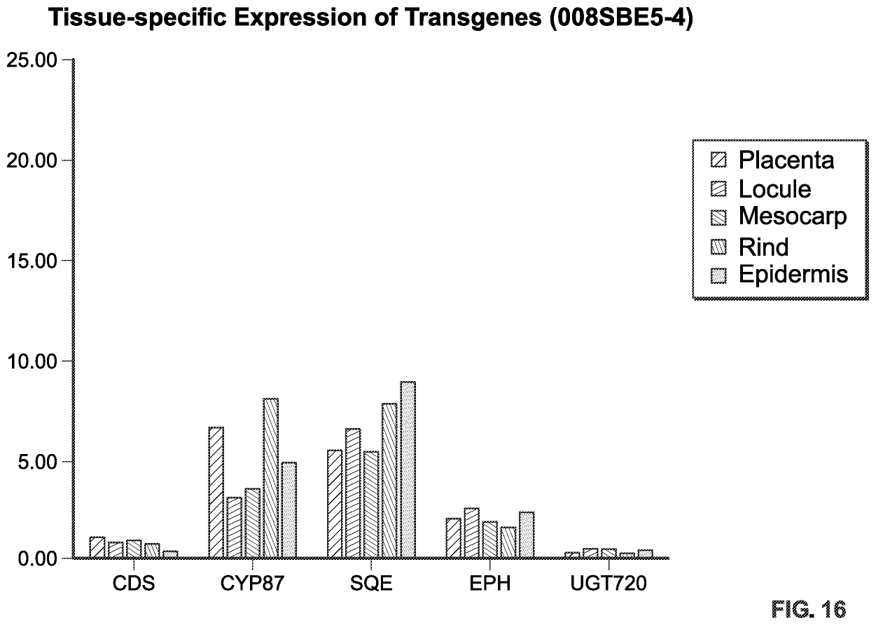

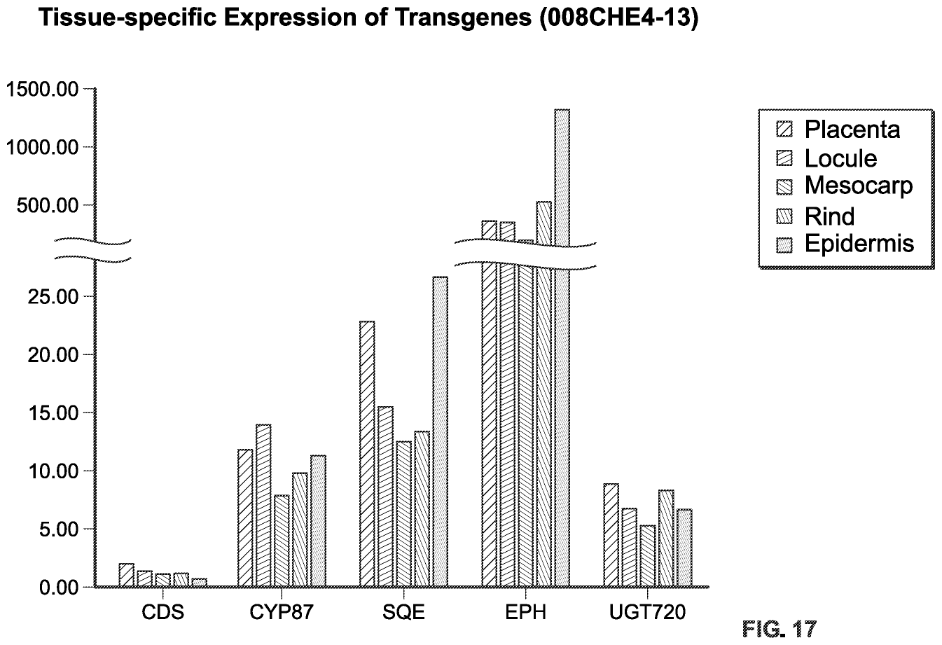

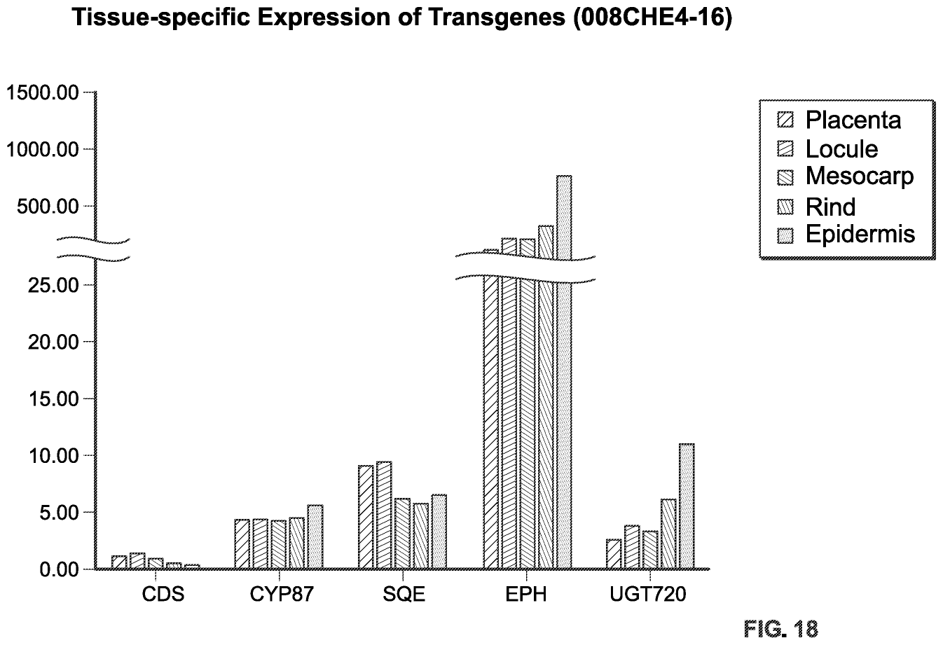

To understand difference in the ability to detect mogroside production in fruit, watermelons from various newly created plant lines were collected and dissected into the various fruit parts. RNA was then extracted from the various fruit part samples, and the RNA expression levels of the various newly integrated pathway genes were quantified using Q-RT-PCR measured using a standard protocol. The measurements showed several trends as illustrated in FIGS. 15-18. As can be seen, all transgenes CDS, CYP87, SQE, EPH, and UGT720 were expressed in all fruit tissues including placenta, locule, mesocarp, rind, and epidermis. It is notable that the expression pattern is consistent among all tissue types. In addition, CDS and UGT720 expression is lower than CYP87, SQE and EPH. EPH is expression dramatically higher in some fruit parts, including the mogroside IIE containing fruit (008CHE4-13). This potentially is due to the positional effects of transgene insertion.

FIGS. 15-18 show the analytical results of various tissues of representative transgenic watermelon samples for the expression of mogroside pathway transgenes. As can be seen, all transgenes CDS, CYP87, SQE, EPH, and UGT720 were expressed in all fruit tissues including placenta, locule, mesocarp, rind, and epidermis. It is notable that the expression pattern is consistent among all tissue types. In addition, CDS and UGT720 expression is lower than CYP87, SQE and EPH. EPH is expression dramatically higher in some fruits.

Metabolic modulation: Same procedure provided in Example 3 was followed to monitor the metabolic modulation and analyze metabolites of transgenic watermelon samples.

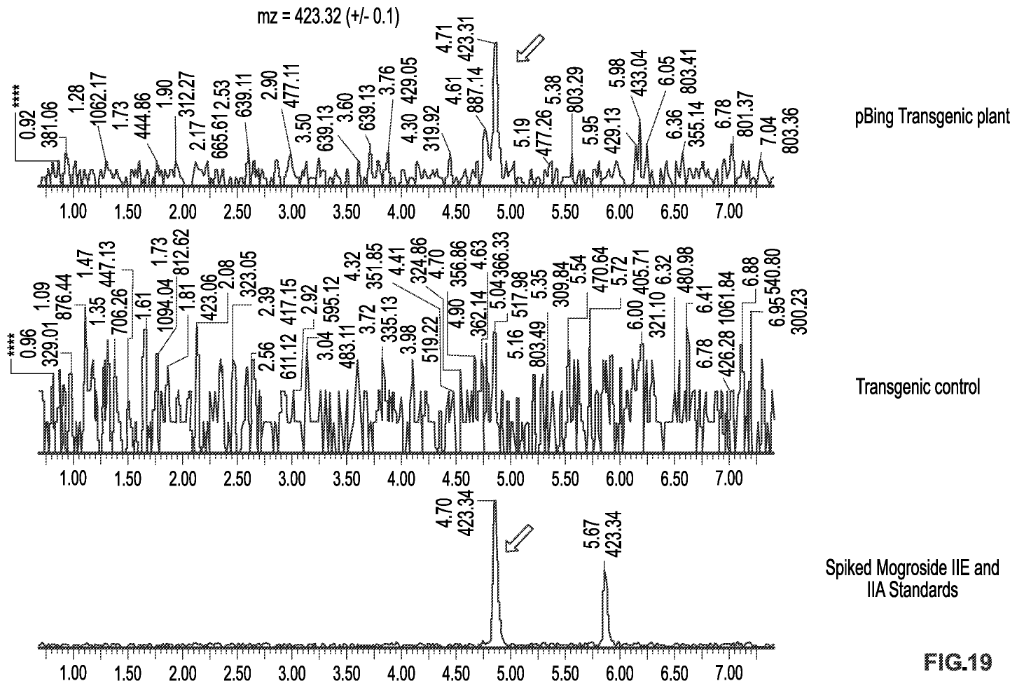

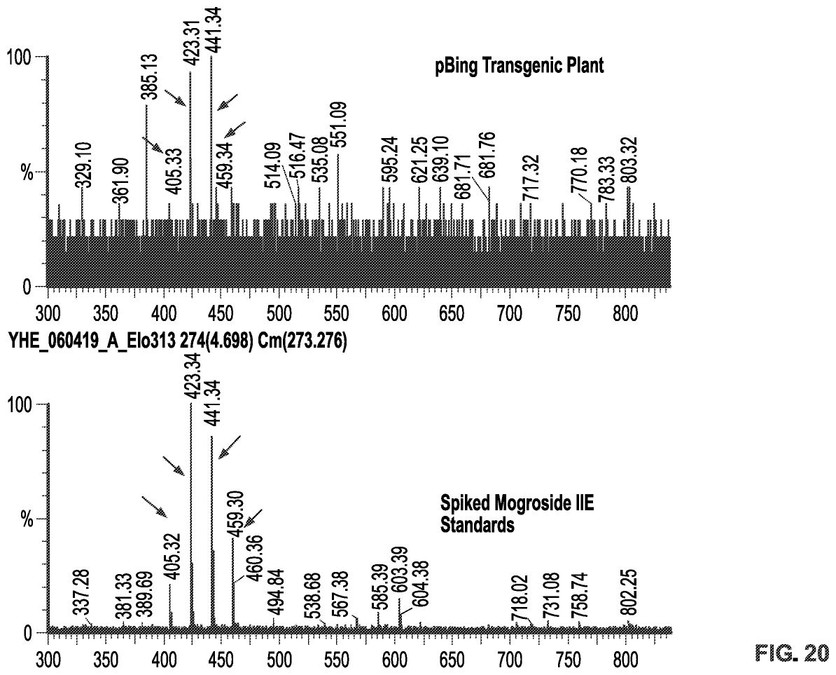

As shown in FIGS. 19-20, comparing with the control, the transgenic watermelons comprising expression cassettes pBing008 showed presence of Mogroside IIE, indicating successful production of mogrosides through the intended enzymatic pathway.

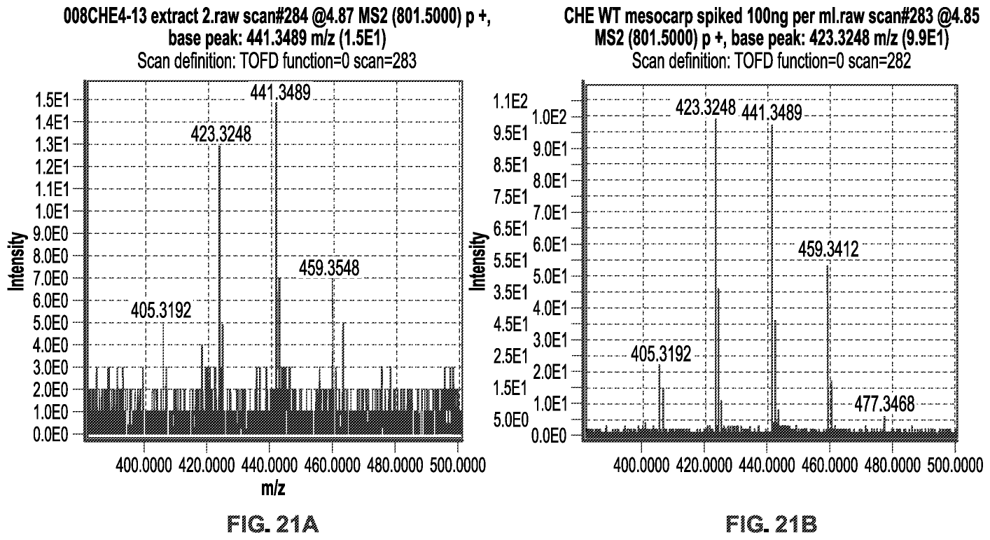

Transgenic plants producing Mogroside IIE were evaluated for their ability to produce Mogroside IIE in fruit. At least one plant was able to produce fruits (008CHE4-13). An extract from the fruits of this plant was made and analyzed by UPLC-TOFMS. The extract of fruits showed the characteristic mass fingerprint for mogroside IIE (shown in FIG. 21a). As a positive control, an extract of the wild type, unmodified fruit was created and spiked with 100 ng/ml Mogroside IIE (shown in FIG. 21b). These surprising results indicate the unexpected capability of the transgenic plants according to the present disclosure of producing fruits comprising non-native mogrosides.

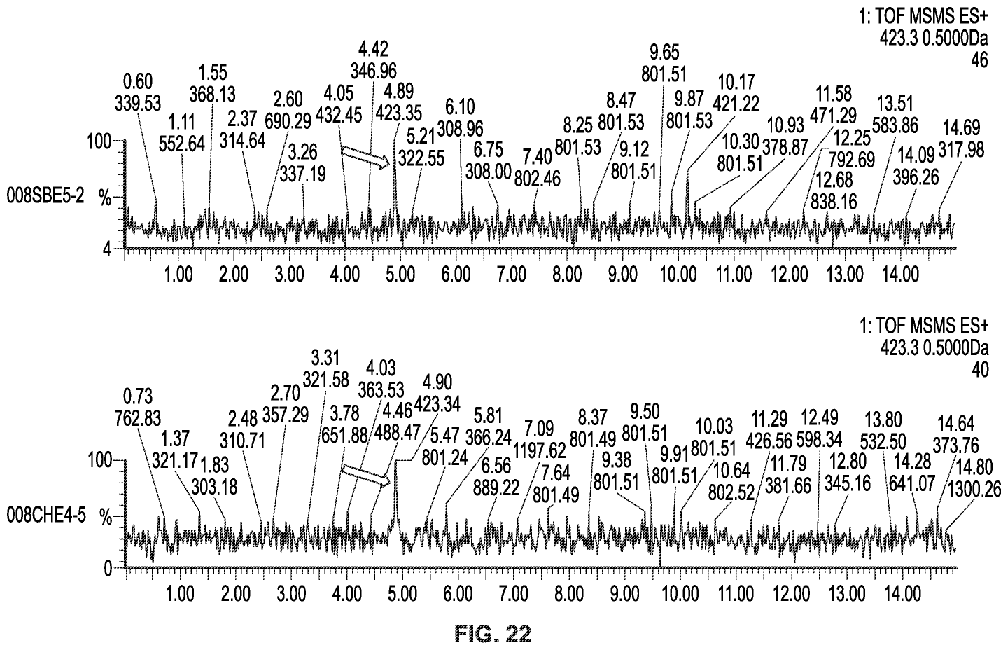

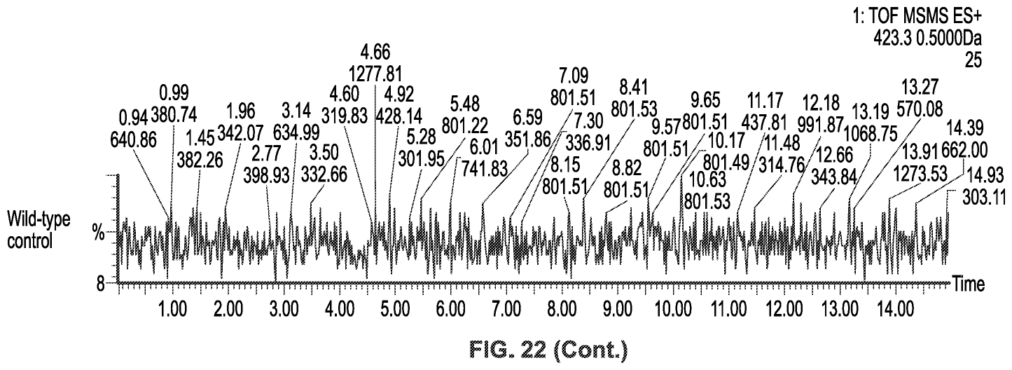

FIG. 22 shows the UPLC-TOFMS results of the seed coats from the transgenic watermelon samples 008SBE5-2 and 008CHE4-5, both comprising the expression cassette pBing008. As can be seen, Mogroside IIE was detected in the seed coat of the fruit, indicating the production of mogrosides in other tissues or parts of the transgenic watermelon, but not limited to the fruit. These surprising results indicate the unexpected reproducibility of the transgenic plants of the present disclosure.

### Example 6: Expression of Mogroside-Producing Transgenes and Production of Mogrosides in Transgenic Watermelon T0 and T1 Plants

A further investigation was carried out to analyze the expression of target transgenes and production of mogrosides in both transgenic watermelons (TO plants) and the progenies thereof (T1 plants).

1. Gene Expression Analysis of Target Genes in T0 Plants

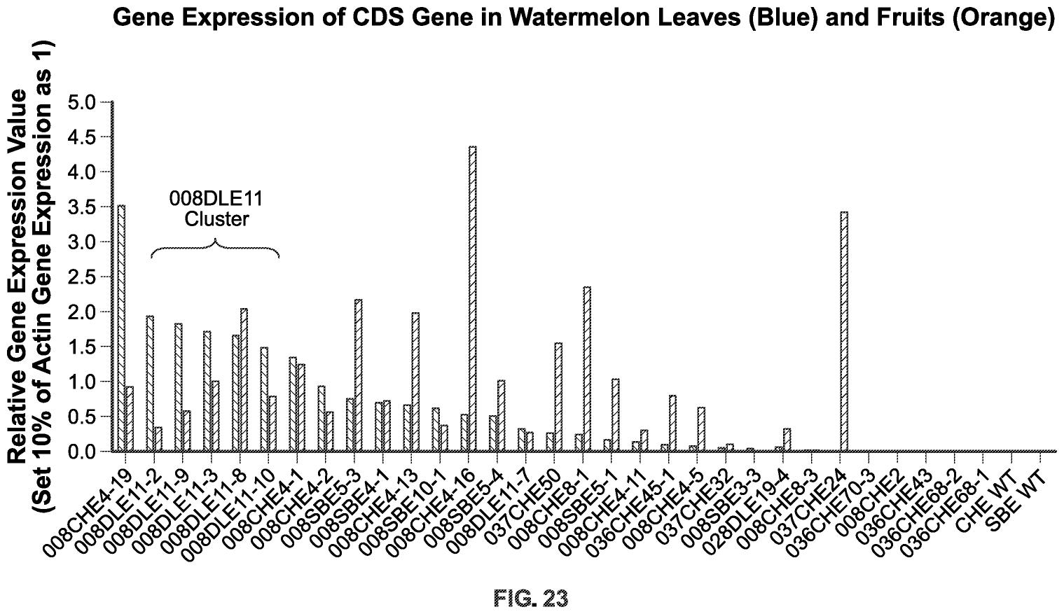

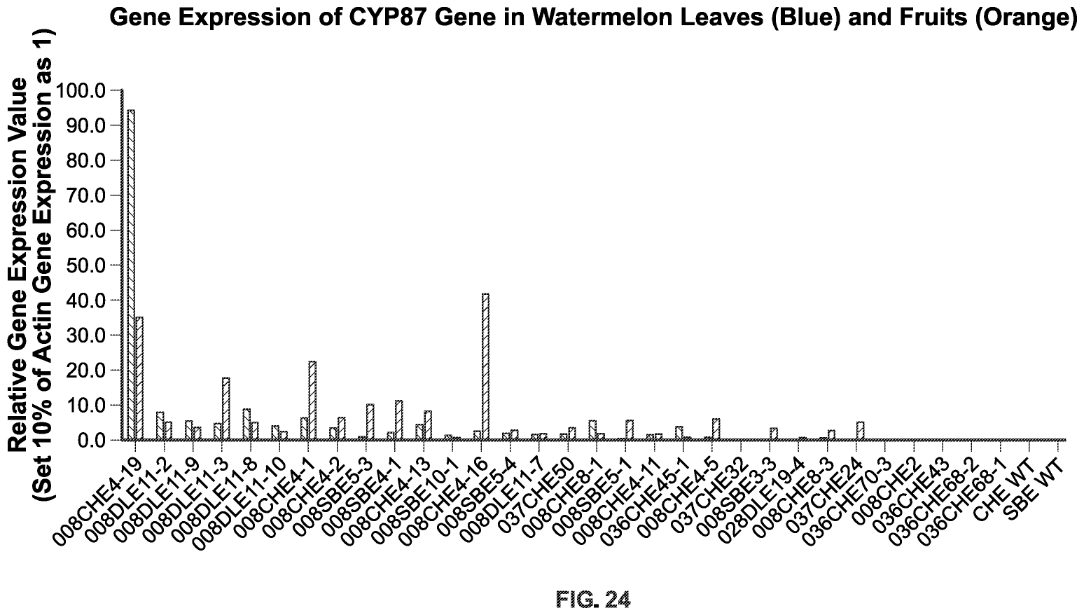

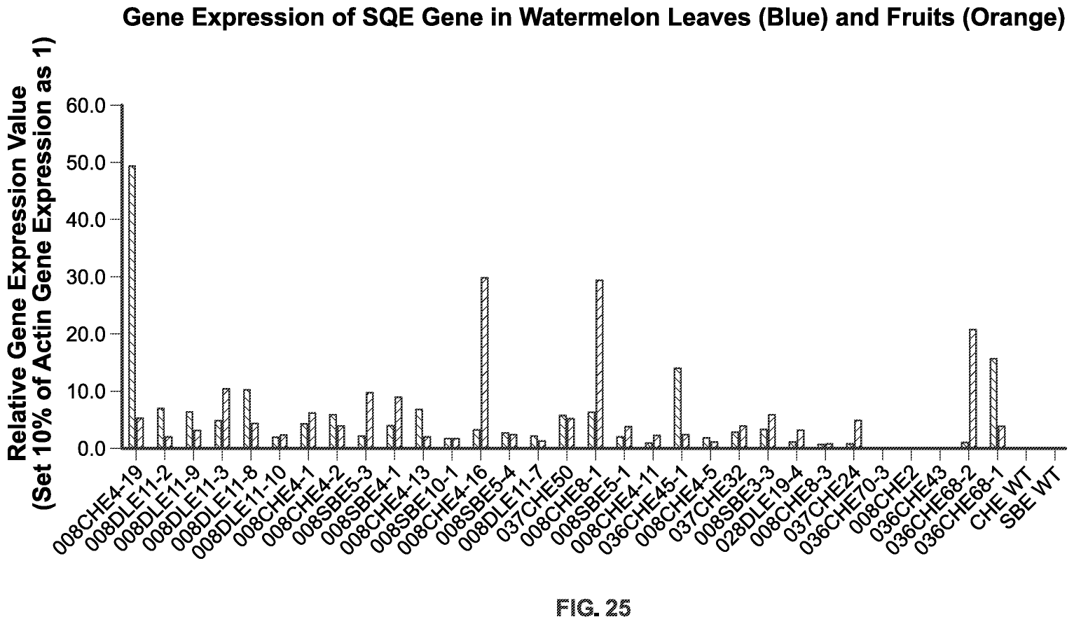

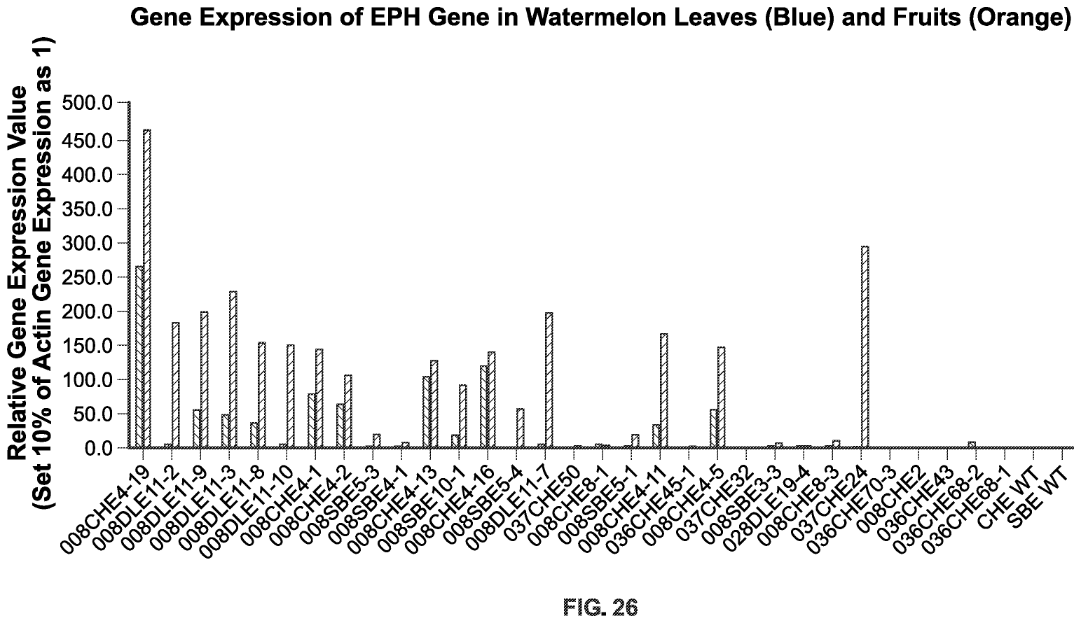

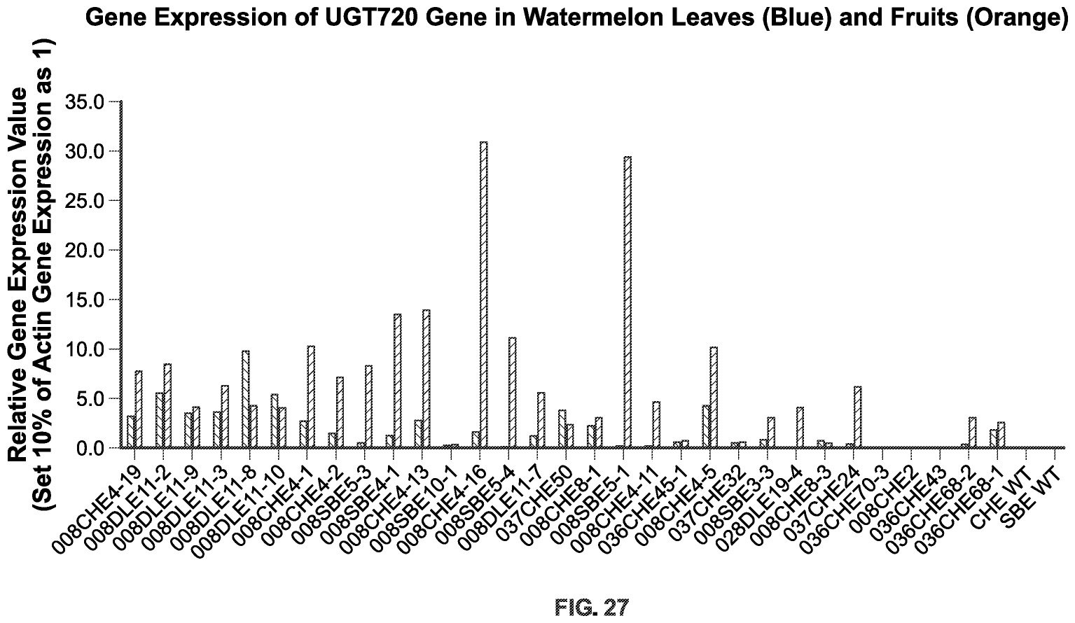

Over 100 transgenic watermelon lines were produced, according to the methods provided in Example 5. Thirty-one plants that produced fruits were used for gene expression analysis. First, the expression level of CDS, a key limiting enzyme in the pathway were studied in leaf and fruit tissues using Q-RT-PCR. To compare gene expression across all samples, all gene expression values were normalized to 10% of Actin expression (set as 1). The results (FIG. 23) showed various expression levels, confirming the presence and expression of the transgene CDS. Although the expression variation seems to be random in leaves, a more consistent pattern can be seen in fruits, where multiple plants from two families of transgenic lines, 008CHE4 and 008DLE11, according to Table 6, showed the highest expression (FIG. 23). 008CHE4-19 showed the highest gene expression in the fruit. Several related transgenic fruits, originally separated from 008DLE11 explant, also showed high CDS expression in fruits. Significant variations were found between leaf and fruit expression. Comparatively, wild type controls (on the right of FIG. 23) showed no CDS expression as expected. Expression levels of other 4 target genes, CYP87, SQE, EPH, and UGT720, were illustrated in FIGS. 24-27, respectively.

2. Metabolic Screening of Transgenic Watermelon TO Fruits Confirming Presence of Mogroside IIE in Transgenic Event 008CHE4-19

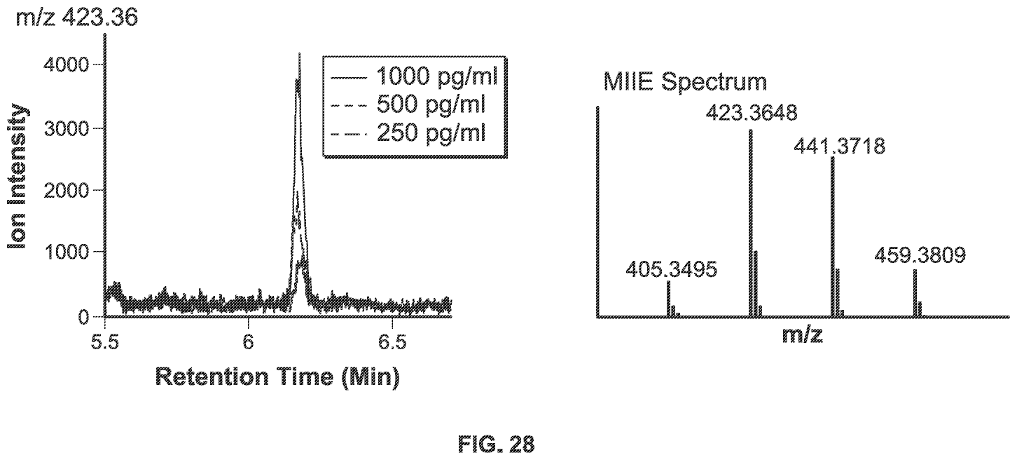

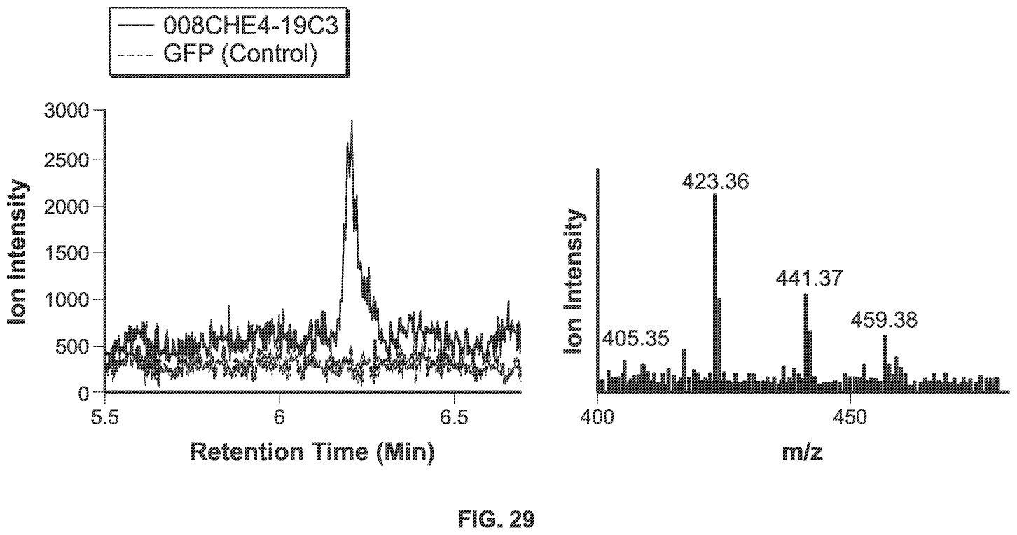

Watermelon fruits were analyzed for mogrol-derived compounds using UPLC-MS, and the results are shown in FIG. 29. As a control, standard Mogroside IIE was also analyzed using UPLC-MS, and the results are shown in FIG. 28. In comparison, results of both the ion chromatogram and the mass spec fragments from 008CHE4-19 TO fruit showed close match to those of Mogroside IIE standard, suggesting biosynthesis and accumulation of Mogroside IIE in this fruit. Interestingly, the fruit from 008CHE4-19 also expressed the highest level of CDS, among all fruits characterized in TO, indicating a possible correlation between CDS expression and MILE biosynthesis in TO watermelon fruits.

3. Event Characterization and Gene Expression Analysis of Transgenes in Progenies of the Transgenic Watermelons (T1 Generation)

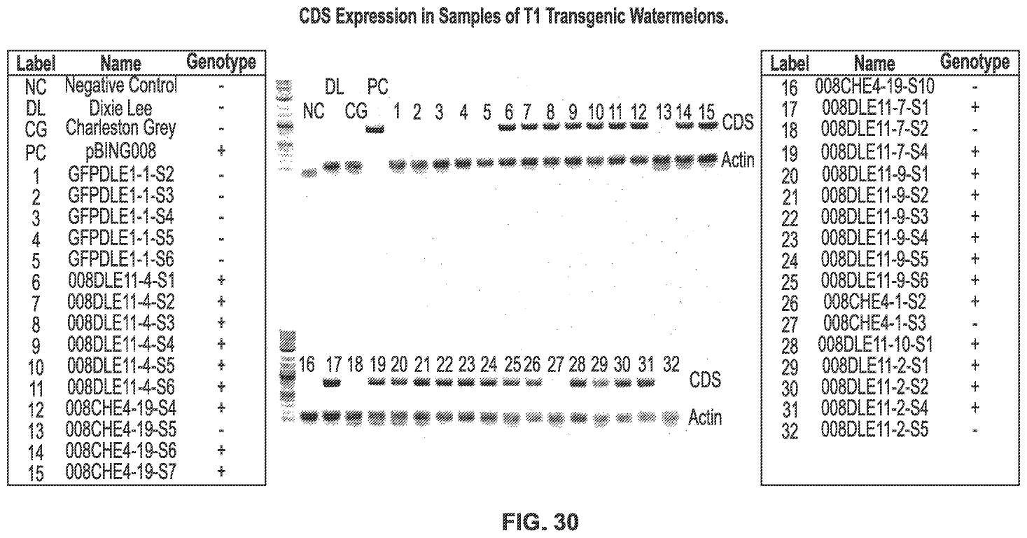

To study the inheritance of Mogroside-producing transgenes and confirm that Mogroside IIE can be produced in more than one generation of transgenic watermelon, seeds from 008CHE4 and 008DLE11 fruits were collected and germinated. After germination, 32 T1 plants (including 5 GFP transgenic controls) were genotyped by PCR, using DNA extracted from leaf tissues. The PCR probes were designed to amplify two targets: the endogenous watermelon actin gene, as positive control, and the CDS gene, as an indication of transgene integration. As shown in FIG. 30, presence of the Actin product band indicates successful PCR from watermelon genomic DNA; and presence of CDS product band indicates presence of the transgenes. The identities and genotyping conclusions of all samples were listed in the table on the left and right. As expected, several wild type segregants were also identified (samples #13, #16, #18, #27 and #32 according to FIG. 30). These genotyping results confirmed presence and successful inheritance of the transgene cassettes into T1 generation of watermelon.

The leaves from transgenic T1 plants were also sampled for Q-RT-PCR detection of all five target genes. The results are summarized in Table 8. Primers were designed to specifically amplify the transgenes, and not the watermelon homologs (as shown by negative controls). The values were averages of three independent biological replicates and normalized to 10% of Actin expression, which was previously set as an expression standard. The plants 008CHE4-1-S3, 008CHE4-19-S5, 008CHE4-19-S10, 008DLE11-2-S5, and 008DLE11-7-S2 were found to be wild type segregants. Consistent with genotyping results, all the wild type segregants and negative, non-transgenic plants showed virtually no expression of these targets. It was found that 008DLE11-2 family shows overall higher expression of all five target genes, compared to other transgenic lines including 008CHE4-19. Among these five genes, EPH, driven by CsVMV promoter, consistently showed a very high expression (about 10 times of actin), suggesting strong activity of this promoter in watermelon.

TABLE 8
Gene expression levels of all five mogroside-producing genes in
leaves of T1 plants of transgenic watermelons.

| Plant ID | CDS | CYP87 | SQE | EPH | UGT720 |
|---|---|---|---|---|---|
| 008CHE4-1-S2 | 0.33 | 0.41 | 0.80 | 125.72 | 0.39 |
| 008CHE4-1-S3 | 0.02 | 0.00 | 0.00 | 0.00 | 0.00 |
| 008CHE4-19-S4 | 0.39 | 0.69 | 3.34 | 75.41 | 0.61 |
| 008CHE4-19-S5 | 0.01 | 0.00 | 0.00 | 0.01 | 0.00 |
| 008CHE4-19-S6 | 0.18 | 0.80 | 3.03 | 39.72 | 0.14 |
| 008CHE4-19-S7 | 0.15 | 0.27 | 1.96 | 43.62 | 0.24 |
| 008CHE4-19-S10 | 0.00 | 0.00 | 0.04 | 0.63 | 0.00 |
| 008DLE11-2-S1 | 0.79 | 8.32 | 16.85 | 65.67 | 0.43 |
| 008DLE11-2-S2 | 0.76 | 1.39 | 5.48 | 106.60 | 1.51 |
| 008DLE11-2-S4 | 0.60 | 1.73 | 6.78 | 107.20 | 1.60 |
| 008DLE11-2-S5 | 0.01 | 0.00 | 0.00 | 0.03 | 0.00 |
| 008DLE11-4-S1 | 0.23 | 0.38 | 3.55 | 5.32 | 0.40 |
| 008DLE11-4-S2 | 0.18 | 0.57 | 4.03 | 51.93 | 0.22 |
| 008DLE11-4-S3 | 0.40 | 0.67 | 4.63 | 16.10 | 0.79 |
| 008DLE11-4-S4 | 0.54 | 0.28 | 26.81 | 66.58 | 1.17 |
| 008DLE11-4-S5 | 0.32 | 0.43 | 4.13 | 139.61 | 0.92 |
| 008DLE11-4-S6 | 0.22 | 0.52 | 2.28 | 96.92 | 0.47 |
| 008DLE11-7-S1 | 0.44 | 0.69 | 2.72 | 135.76 | 1.09 |
| 008DLE11-7-S2 | 0.00 | 0.00 | 0.01 | 0.04 | 0.00 |
| 008DLE11-7-S4 | 0.36 | 0.84 | 3.08 | 103.78 | 0.70 |
| 008DLE11-9-S1 | 0.55 | 6.76 | 9.09 | 68.27 | 0.30 |
| 008DLE11-9-S2 | 0.17 | 0.41 | 2.73 | 86.33 | 1.50 |
| 008DLE11-9-S3 | 0.20 | 0.48 | 1.71 | 99.63 | 0.58 |
| 008DLE11-9-S4 | 0.58 | 1.07 | 4.01 | 43.07 | 0.69 |
| 008DLE11-9-S5 | 0.47 | 0.83 | 3.10 | 91.89 | 0.71 |
| 008DLE11-9-S6 | 0.22 | 3.36 | 11.00 | 70.62 | 0.27 |
| 008DLE11-10-S1 | 0.42 | 0.72 | 4.13 | 1116.70 | 0.67 |
| GFPDLE-1-S6 | 0.00 | 0.00 | 0.00 | 0.00 | 0.00 |
| CHE Wild Type | 0.00 | 0.00 | 0.00 | 0.00 | 0.00 |
| SBE Wild Type | 0.00 | 0.00 | 0.00 | 0.00 | 0.00 |
| DLE Wild Type | 0.00 | 0.00 | 0.00 | 0.00 | 0.00 |

4. Metabolic Analysis Identified Three Watermelon T1 Fruits that Produced Mogrosides

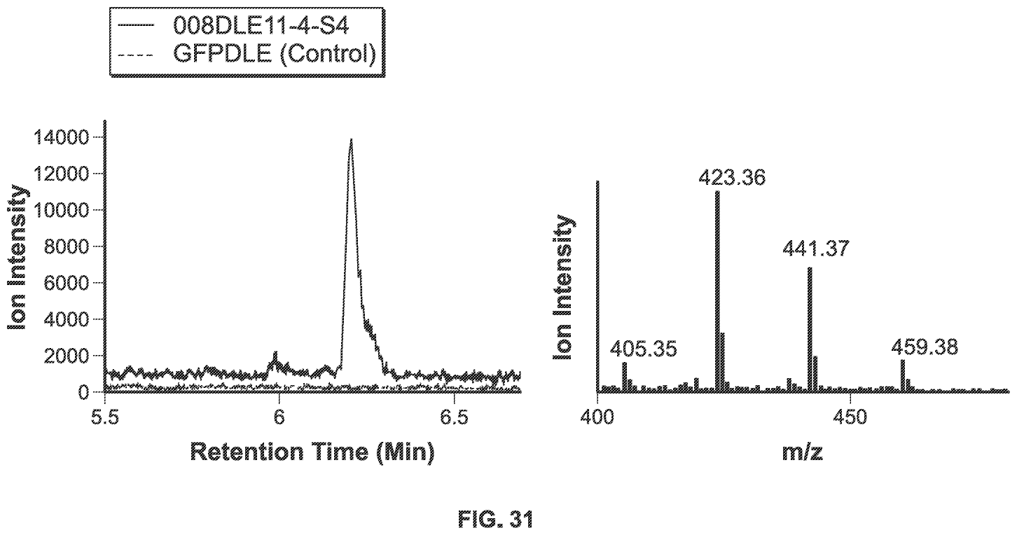

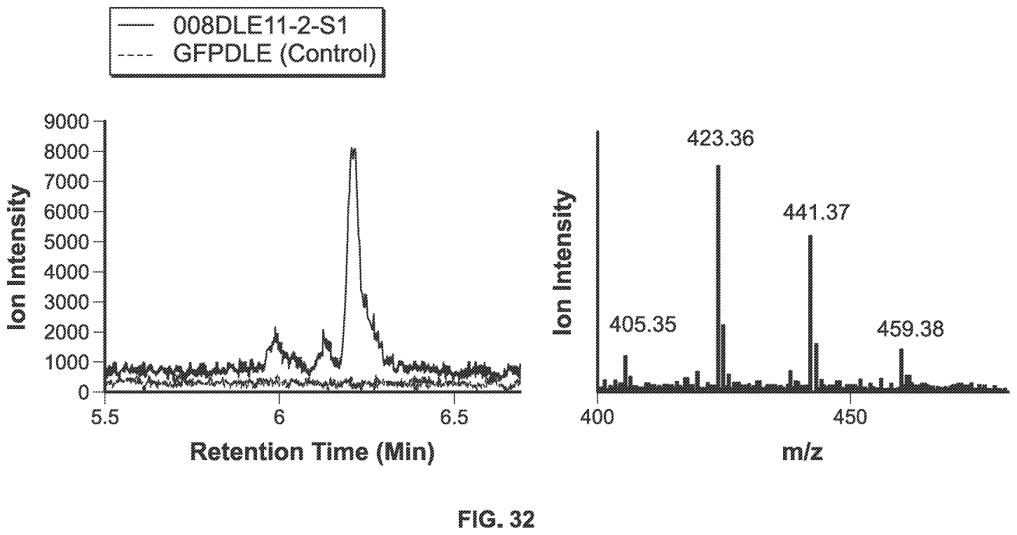

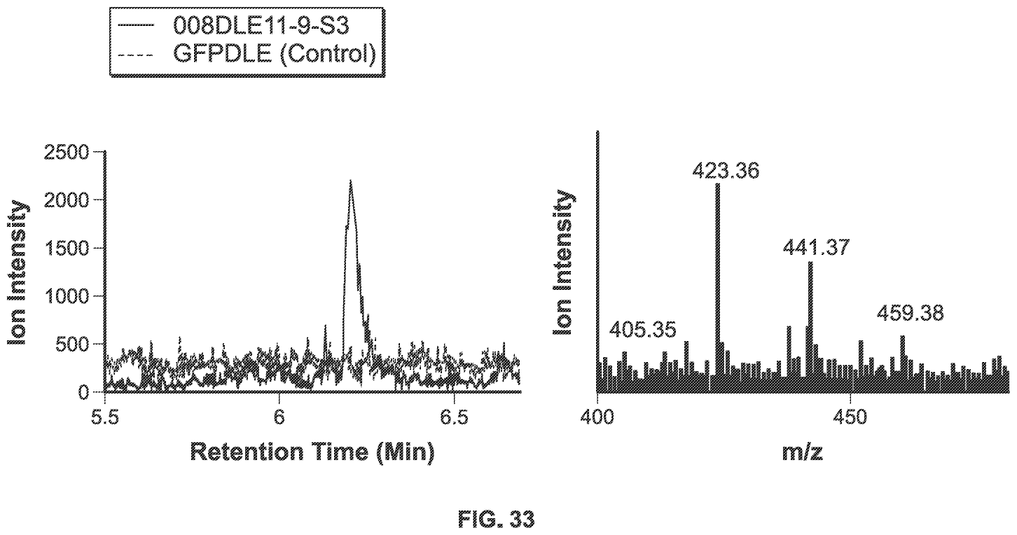

Fruits produced from T1 lines were harvested for metabolic analysis. The LC-MS results, as shown in FIGS. 31-33, confirmed presence of Mogroside IIE in 008DLE11-4-54, 008DLE11-2-S1, and 008DLE11-9-53, all of which are the progenies of the TO transgenic watermelons comprising the expression cassette pBing008. Quantitively, these three T1 samples produced about 30 ng, about 17 ng, and about 3 ng (per gram dry weight) of Mogroside IIE, respectively.

The following numbered clauses define further example aspects and features of the present disclosure:

1. A plant comprising a genomic transformation event, wherein the genomic transformation event produces a non-native expression or concentration of mogroside pathway enzyme(s), wherein the plant biosynthetically produces non-native mogrol precursors, mogrol, mogrosides, and/or metabolites or derivatives thereof.

2. A plant comprising non-native mogrol precursors and/or mogrol, wherein the plant biosynthetically produces mogrosides, and/or metabolites or derivatives thereof.

3. The plant of clause 1, wherein the plant is a transgenic plant and wherein the genomic transformation event comprises an expression cassette, wherein the expression cassette comprises one or more of the nucleotide sequences as set forth in SEQ ID

NOs: 1-31.

4. The plant of clause 2, wherein the plant is a transgenic plant comprising an expression cassette, wherein the expression cassette comprises one or more of the nucleotide sequences as set forth in SEQ ID NOs: 1-31.

5. The transgenic plant of any of clauses 3-4, wherein the expression cassette comprises one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

6. The transgenic plant of clause 2, wherein the mogroside pathway enzyme(s) has a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

7. The transgenic plant of any of the clauses 3-6, wherein the expression cassette comprises one or more sequences selected from the group consisting of: promoter, spacer, epitope tag, terminator, reporter gene, or combinations thereof.

8. The transgenic plant of clause 1, wherein the mogroside pathway enzyme(s) is selected from the group consisting of: circubitadienol synthase (CDS), squalene epoxidase (SQE), epoxy hydrolase (EPH), cytochrome P450, uridine-5′-diphospho (UDP) dependent glucosyltransferase (UGT), or combinations thereof.

9. The transgenic plant of any of clauses 1-8, wherein the mogroside is selected from the group consisting of Siamenoside I, Siratose, Mogroside VI, Mogroside V, Isomogroside V, Mogroside IV, Mogroside III, Mogroside IIIE, Mogroside II, Mogroside IIA, Mogroside IIA1, Mogroside IIA2, Mogroside IIE, MogrosideIIE2, Mogroside I, Mogroside IA, Mogroside IE, or any combinations thereof.

10. The transgenic plant of any of clauses 1-9, wherein the mogroside is selected from the group consisting of Mogroside IA, Mogroside IE, Mogroside IIA, Mogroside IIA1, Mogroside IIA2, Mogroside IIE, Mogroside 11E2, or any combinations thereof.

11. A plant part obtainable from the plant according to any of clauses 1-10, including but not limiting to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant, wherein the plant part comprises mogrol precursors, mogrol, mogroside, and/or metabolites or derivatives thereof.

12. A plant according to any of clauses 1-11, wherein a progeny or an ancestor thereof is a source of non-native enzyme(s) enabling the progeny and the ancestor to produce mogrol precursors, mogrol, mogrosides, and/or metabolites or derivatives thereof.

13. The plant of any of clauses 1-12, wherein the plant a diploid plant.

14. The plant of any of clauses 1-13, wherein the plant is Cucurbitaceae/Curcubits.

15. The plant of any of clauses 1-14, wherein the plant is Citrullus lanatus (watermelon).

16. A mogroside sweetener derived from a plant, wherein the plant or a part thereof biosynthetically produces and comprises non-native mogrol precursors, mogrol, mogrosides, and/or metabolites or derivatives thereof.

17. The mogroside sweetener of clause 16, wherein the sweetener is in an extract of the plant.

18. The mogroside sweetener of any of clauses 16-17, wherein the sweetener is purified from the plant or a part thereof.

19. The mogroside sweetener of clause 18, wherein the sweetener is purified by extraction, steeping, chromatography, or absorption chromatogram.

20. A food, ingredient, flavor, or beverage comprising the sweetener of any of the clauses 16-19.

21. A biosynthetic method for producing non-native mogrol precursors, mogrol, or mogrosides, comprising the steps of:
(a) combining a plant with a genomic transformation event thereby forming a transgenic plant, wherein the genomic transformation event produces a non-native expression or concentration of mogrol/mogroside pathway enzyme(s);
(b) growing and regenerating a population of the transgenic plant of (a);
(c) selecting the transgenic plants that produce mogrosides; and
(d) harvesting mogrosides.

22. The method of clause 21 further comprising:
preparing/providing plasmids comprising an expression cassette, wherein the expression cassette expresses non-native mogrol/mogroside pathway enzyme(s);
transforming a host cell with the plasmids; and
transfecting the plant with a plurality of the transformed host cell.

23. A method of making a plant producing non-native mogrol precursors, mogrol, or mogrosides, comprising combining a plant with a genomic transformation event thereby forming the transgenic plant, wherein the genomic transformation event produces a non-native expression or concentration of mogrol/mogroside pathway enzyme(s).

24. The method of clause 23, wherein combining the plant with the genomic transformation event is performed using one or more of the following methods: use of liposomes, use of electroporation, use of chemicals that increase free DNA uptake, use of injection of the DNA directly into the plant, use of particle gun bombardment, use of microprojection, or use of Agrobacterium-mediated transformation.

25. The method of clauses 23-24 further comprising:
preparing/providing plasmids comprising an expression cassette, wherein the expression cassette expresses non-native mogrol/mogroside pathway enzyme(s);
transforming a host cell with the plasmids; and
transfecting the plant with a plurality of the transformed host cell.

26. The method of clause 25, wherein the host cell is a microorganism.

27. The method of clause 26, wherein the microorganism is selected from the group consisting of plant cell, mammalian cell, insect cell, fungal cell, algal cell, bacterial cell, or combinations thereof.

28. The method of clause 27, wherein the bacterial cell is a gram-negative bacterium.

29. The method of clause 28, wherein, the gram-negative bacterium is Agrobacterium Tumefaciens.

30. The biosynthetic method of clause 29, wherein the host cell is transformed with the plasmids using free-thaw method.

31. A biosynthetic method for producing non-native mogrol precursors, mogrol, or mogrosides, comprising the steps of:
(a) combining a plant with a genomic transformation event thereby forming a gene-edited plant, wherein the genomic transformation event produces a non-native expression or concentration of mogrol/mogroside pathway enzyme(s);
(b) growing and regenerating a population of the gene-edited plant of (a);
(c) selecting the gene-edited plants that produce mogrosides; and
(d) harvesting mogrosides.

32. The method of clause 32 further comprising:
preparing/providing plasmids comprising an expression cassette, wherein the expression cassette expresses non-native mogrol/mogroside pathway enzyme(s);
transforming a host cell with the plasmids; and
transfecting the plant with a plurality of the transformed host cell.

33. A food, ingredient, flavor, or beverage comprising the sweetener of any of the clauses 21-32.

34. The plant of clause 1, wherein the plant is a gene-edited plant and wherein the genomic transformation event is added to the plant by a method selected from a group comprising transcription activator-like effector nucleases (TALENs), zinc-finger nucleases (ZFNs), meganucleases (MNs) and combinations thereof.

The above specification, examples and data provide a complete description of the manufacture and use of the composition of the invention. Since many embodiments of the invention can be made without departing from the spirit and scope of the invention, the invention resides in the claims hereinafter appended.

1. A plant comprising a genomic transformation event, wherein the genomic transformation event produces a non-native expression or concentration of mogroside pathway enzyme(s), wherein the plant and/or a progeny and/or a seed thereof biosynthetically produce non-native mogrol precursors, mogrol, mogrosides, and/or metabolites or derivatives thereof.

2. A plant comprising non-native mogrol precursors and/or mogrol, wherein the plant and/or a progeny and/or seed thereof biosynthetically produce mogrosides, and/or metabolites or derivatives thereof.

3. The plant of claim 1, wherein the plant is a transgenic plant and wherein the genomic transformation event comprises an expression cassette, wherein the expression cassette comprises one or more of the nucleotide sequences as set forth in SEQ ID NOs: 1-31.

4. The plant of claim 2, wherein the plant is a transgenic plant comprising an expression cassette, wherein the expression cassette comprises one or more of the nucleotide sequences as set forth in SEQ ID NOs: 1-31.

5. The plant of any of claims 3-4, wherein the expression cassette comprises one or more of the nucleotide sequences having a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

6. The plant of claim 1, wherein the mogroside pathway enzyme(s) has a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-31.

7. The plant of any of the claims 3-6, wherein the expression cassette comprises one or more sequences selected from the group consisting of: promoter, spacer, epitope tag, terminator, reporter gene, or combinations thereof.

8. The plant of claim 1, wherein the mogroside pathway enzyme(s) is selected from the group consisting of: circubitadienol synthase (CDS), squalene epoxidase (SQE), epoxy hydrolase (EPH), cytochrome P450, uridine-5′-diphospho (UDP) dependent glucosyltransferase (UGT), or combinations thereof.

9. The plant of claim 1, wherein the mogroside is selected from the group consisting of Siamenoside I, Siratose, Mogroside VI, Mogroside V, Isomogroside V, Mogroside IV, Mogroside III, Mogroside IIIE, Mogroside II, Mogroside IIA, Mogroside IIA1, Mogroside IIA2, Mogroside IIE, MogrosideIIE2, Mogroside I, Mogroside IA, Mogroside IE, or any combinations thereof.

10. The plant of claim 1, wherein the mogroside is selected from the group consisting of Mogroside IA, Mogroside IE, Mogroside IIA, Mogroside IIA1, Mogroside IIA2, Mogroside IIE, Mogroside 11E2, or any combinations thereof.

11. A plant part obtainable from the plant according to claim 1, including but not limiting to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the transgenic plant, wherein the plant part comprises mogrol precursors, mogrol, mogroside, and/or metabolites or derivatives thereof.

12. A plant according to claim 1, wherein a progeny or an ancestor thereof is a source of non-native enzyme(s) enabling the progeny and the ancestor to produce mogrol precursors, mogrol, mogrosides, and/or metabolites or derivatives thereof.

13. The plant of claim 1, wherein the plant is Cucurbitaceae/Curcubits.

14. A mogroside sweetener derived from a plant of claim 1, wherein the plant or a part thereof biosynthetically produces and comprises non-native mogrol precursors, mogrol, mogrosides, and/or metabolites or derivatives thereof.

15. A food, ingredient, flavor or beverage comprising the sweetener of claim 1.

16. A biosynthetic method for producing non-native mogrol precursors, mogrol, or mogrosides, comprising the steps of:
(a) combining a plant with a genomic transformation event thereby forming a plant, wherein the genomic transformation event enables the plant and/or a progeny and/or a seed thereof to produce a non-native expression or concentration of mogrol or mogroside pathway enzyme(s);
(b) growing and regenerating a population of the plant of (a);
(c) selecting the transgenic plants that produce mogrol or mogrosides; and
(d) harvesting mogrol or mogrosides.

17. The method of claim 16 further comprising:
preparing/providing plasmids comprising an expression cassette, wherein the expression cassette expresses non-native mogrol or mogroside pathway enzyme(s);
transforming a host cell with the plasmids; and
transfecting the plant with a plurality of the transformed host cell.

18. A method of making a plant producing non-native mogrol precursors, mogrol, or mogrosides, comprising combining a plant with a genomic transformation event, wherein the genomic transformation event e enables the plant and/or a progeny and/or a seed thereof to produce a non-native expression or concentration of mogrol/mogroside pathway enzyme(s).

19. The method of claim 18 further comprising:
preparing/providing plasmids comprising an expression cassette, wherein the expression cassette expresses non-native mogrol/mogroside pathway enzyme(s);
transforming a host cell with the plasmids; and
transfecting the plant with a plurality of the transformed host cell.

20. The method of claim 19, wherein, the host cell is Agrobacterium Tumefaciens.

21. The plant of claim 1, wherein the plant is a gene-edited plant and wherein the genomic transformation event is added to the plant by a method selected from a group comprising transcription activator-like effector nucleases (TALENs), zinc-finger nucleases (ZFNs), meganucleases (MNs) and combinations thereof.


SEQUENCE LISTING (31 sequences)

SEQ ID NO: 1 - DNA, 2277 bp, Siraitia grosvenorii
atgtggaggttaaaggtcggagcagaaagcgttggggagaatgatgagaaatggttgaagagcataagcaatcacttgggacgccaggtgtgggagttctgtccggatgccggcacccaacaacagctcttgcaagtccacaaagctcgtaaagcgttccacgatgaccgtttccaccgaaagcaatcttccgatctctttatcactattcagtatggaaaggaagtagaaaatggtggaaagacagcgggagtgaaattgaaagaaggggaagaggtgaggaaagaggcagtagagagtagcttagagagggcattaagtttctactcaagcatccagacaagcgatgggaactgggcttcggatcttggggggcccatgtttttacttccgggtctggtgattgccctctacgttacaggcgtcttgaactctgttttatccaagcaccaccggcaagagatgtgcagatatgtttacaatcaccagaatgaagatggggggtggggactccacatcgagggcccaagcaccatgtttggttccgcactgaattatgttgcactcaggctgcttggagaagacgccaacgccggggcaatgccaaaagcacgtgcttggatcttggaccacggtggcgccaccggaatcacttcctggggcaaattgtggctttctgtacttggagtctacgaatggagtggcaataatcctcttccacccgaattttggttatttccttacttcctaccatttcatccaggaagaatgtggtgccattgtcgaatggtttatctaccaatgtcatacttatatggaaagagatttgttgggccaatcacacccatagttctgtctctcagaaaagaactctacgcagttccatatcatgaaatagactggaataaatctcgcaatacctgtgcaaaggaggatctgtactatccacatcccaagatgcaagatattctgtggggatctctccaccacgtgtatgagcccttgtttactcgttggcctgccaaacgcctgagagaaaaggctttgcagactgcaatgcaacatattcactatgaagatgagaatacccgatatatatgccttggccctgtcaacaaggtactcaatctgctttgttgttgggttgaagatccctactccgacgccttcaaacttcatcttcaacgagtccatgactatctctgggttgctgaagatggcatgaaaatgcagggttataatgggagccagttgtgggacactgctttctccatccaagcaatcgtatccaccaaacttgtagacaactatggcccaaccttaagaaaggcacacgacttcgttaaaagttctcagattcagcaggactgtcctggggatcctaatgtttggtatcgtcacattcataaaggtgcatggccattttcaactcgcgatcatggatggctcatctctgactgtacagcagagggattaaaggctgctttgatgttatccaaacttccatccgaaacagttggggaatcattagaacggaatcgcctttgcgatgctgtaaacgttctcctttctttgcaaaacgataatggtggctttgcatcatatgagttgacaagatcatacccttggttggagttgatcaaccccgcagaaacgtttggagatattgtcattgattatccgtatgtggagtgcacctcagccacaatggaagcactgacgttgtttaagaaattacatcccggccataggaccaaagaaattgatactgctattgtcagggcggccaacttccttgaaaatatgcaaaggacggatggctcttggtatggatgttggggggtttgcttcacgtatgcggggtggtttggcataaagggattggtggcagctggaaggacatataataattgccttgccattcgcaaggcttgcgattttttactatctaaagagctgcccggcggtggatggggagagagttacctttcatgtcagaataaggtatacacaaatcttgaaggaaacagaccgcacctggttaacacggcctgggttttaatggccctcatagaagctggccaggctgagagagatccaacaccattgcatcgtgcagcaaggttgttaatcaattcccagttggagaatggtgatttcccccaacaggagatcatgggagtctttaataaaaattgcatgatcacatatgctgcataccgaaacatttttcccatttgggctcttggagagtattgccatcgggttttgactgaa

SEQ ID NO: 2 - DNA, 1420 bp, Siraitia grosvenorii
atgtggactgtcgtgctcggtttggcgacgctgtttgtcgcctactacatccattggattaacaaatggagagattccaagttcaacggagttctgccgccgggcaccatgggtttgccgctcatcggagagacgattcaactgagccgacccagtgactccctcgacgttcaccctttcatccagaaaaaagttgaaagatacgggccgatcttcaaaacctgtctggccggaaggccggtggtggtgtcggcggacgcagagttcaacaactacataatgcttcaggaaggaagagcagtggaaatgtggtatttggatacgctctccaaatttttcggcctcgacaccgagtggctcaaagctctgggcctcatccacaagtacatcagaagcattactctcaatcacttcggcgccgaggccctgcgggagagatttcttccttttattgaagcatcctccatggaagcccttcactcctggtctactcaacctagcgtcgaagtcaaaaatgcctccgctctcatggtttttaggacctcggtgaataagatgttcggtgaggatgcgaagaagctatcgggaaatatccctgggaagttcacgaagctgctaggaggatttctcagtttaccactgaattttcccggcaccacctaccacaaatgcttgaaggatatgaaggaaatccagaagaagctaagagaggttgtagacgatagattggctaatgtgggccctgatgtggaagatttcttggggcaagcccttaaagataaggaatcagagaagttcatttcagaggagttcatcatccaactgttgttttctatcagttttgccagctttgagtccatctccaccactcttactttgattctcaagctccttgatgaacacccagaagtagtgaaagagttggaagctgaacacgaggcgattcgaaaagctagagcagatccagatggaccaattacttgggaagaatacaaatccatgacttttacattacaagtcatcaatgaaaccctaaggttggggagtgtcacacctgccttgttgaggaaaacagttaaagaccttcaagtaaaaggatacataatcccggaaggatggacaataatgcttgtcaccgcttcacgtcacagagatccaaaagtctataaggaccctcatatcttcaatccatggcgttggaaggacttggactcaattaccatccaaaagaacttcatgccttttgggggaggcttaaggcattgtgctggtgctgagtactctaaagtctacttgtgcaccttcttgcacatcctctgtaccaaataccgatggaccaaacttgggggaggaacgattgcaagagcgcatatattgagttttgaagatgggttacacgtgaagttcacacccaaggaag

SEQ ID NO: 3 - DNA, 948 bp, Siraitia grosvenorii
atggaccagattgagcatatcaccatcaacaccaatggcatcaaaatgcacattgcctctgtagggacgggcccagtagttcttcttctgcatggcttcccggaactctggtattcatggcgccaccagcttctgtatctttcttccgtaggatatcgagctattgcgccggacctccgcggctatggcgacacggactcgccggcgtctcctacctcctacaccgctctccacatcgtcggcgatttggttggggctctggacgagcttgggatcgagaaggtgtttctggtcggacatgactggggggcgatcatcgcctggtacttttgcttgttcaggcccgatagaatcaaggcgctggtgaatctgagcgtccagttcatacccagaaacccagcgattcctttcattgagggtttcagaactgcgttcggtgatgacttctatatgtgcaggtttcaggttccaggagaggcagaagaagattttgcctccatcgacacagctcagctgttcaagacatcattatgtaatagaagttctgcacctccatgcttgcctaaagaaattggatttcgtgcgatcccacctccagagaaccttccttcttggctgacagaagaagatatcaactattatgctgccaaatttaagcagacaggcttcaccggagcgttgaactactatcgagcttttgacctaacttgggaactcacggcgccttggacaggagcacagattcaggttccggtgaagttcatcgtcggggattcggatctaacttaccattttccgggagccaaggaatatatccataatggcggattcaaaaaggacgtgccgttgctggaggaagtagttgtagtaaaagatgcttgtcacttcatcaaccaagaaaggccacaagaaatcaatgctcacatccatgacttcatcaataaattc

SEQ ID NO: 4 - DNA, 1584 bp, Siraitia grosvenorii
atggtggatcagtgcgcgttgggatggatcttggcctccgcgctgggcctcgtaattgcgctttgtttcttcgtggctccgaggaggaatcacagaggagtggattcgaaggagagggacgagtgcgtccaaagcgctgcaaccacgaagggagaatgcagattcaacgatcgcgacgttgacgttatcgtcgttggcgccggtgttgccggttccgctcttgctcacactcttggcaaggatggtcgtcgagttcatgtaattgaaagagacttgacagagcctgacagaatcgttggtgaattattacaacctgggggttacctcaaattgattgaattaggacttcaagactgcgtcgaggagattgatgctcaaagggtgtatggctacgcccttttcaaggatggaaagaacactcgactctcttacccattggaaaattttcactctgatgtatctggaaggagctttcacaacgggcgcttcatacagagaatgagggagaaggctgcttcccttcccaatgtcagattggagcaagggacagttacttcgctgcttgaagaaaagggaacgatcaaaggtgtgcagtataagtctaaaaatggtgaagagaaaacagcatatgcacctctgaccattgtttgtgatggctgcttctcaaacttgcgccgctctctctgcaaccctatggttgatgttccctcttattttgtgggattagttcttgagaattgtgagcttccttttgcaaatcacgggcacgttatcctcggagatccttctcccattttattctaccagattagcaggaccgagatccgttgtttggttgatgttcctggtcagaaggttccttctatagcaaatggtgaaatggagaaatatttgaagactgtagtagctcctcaggttcccccgcaaatctacgattcctttatcgctgctatcgacaagggtaatataaggacaatgccaaacagaagcatgcctgctgctccccacccaacgcccggtgccttactgatgggtgatgctttcaacatgcgccaccctcttaccggtggaggaatgaccgtagcattgtctgatatagttgtattgcggaacctcctcaagcctctgaaggacttgagtgatgcatctaccctctgcaagtatcttgaatccttttacactttgcgaaagccagtggcttcgaccatcaacacattggcaggggcattatacaaggtcttttgtgcatcaccagatcaagctaggaaggaaatgcgacaagcctgcttcgattacttgagccttggaggaatattctcaaatggacctgtctccttgctttcagggttgaatcctcgccccttaagtttggttctccatttctttgccgtcgcgatatacggagttggtcgcttattacttccatttccttcagtgaaaggcatctggattggagctagattgatctatagcgcatcaggtatcatattcccaattatacgggcggaaggagttagacagatgttcttccctgcaactgttcctgcttattatagaagtccaccagtgtttaaacccatagta

SEQ ID NO: 5 - DNA, 1422 bp, Siraitia grosvenorii
atggtgcaacctcgggtactgctgtttcctttcccggcactgggccacgtgaagcccttcttatcactggcggagctgctttccgacgccggcatagacgtcgtcttcctcagcaccgagtataaccaccgtcggatctccaacactgaagccctagcctcccgcttcccgacgcttcatttcgaaactataccggatggcctgccgcctaatgagtcgcgcgctcttgccgacggcccactgtatttctccatgcgtgagggaactaaaccgagattccggcaactgattcaatctcttaacgacggtcgttggcccatcacctgtattatcactgacatcatgttatcttctccgattgaagtagcggaagaatttgggattccagtaattgccttctgcccatgcagtgctcgctacttatcgattcacttttttataccgaagctcgttgaggaaggtcaaattccatacgcagatgacgatccgattggagagatccagggggtgcccttgttcgaaggtcttttgcgacggaatcatttgcctggttcttggtctgataaatccgcagatatatctttctcgcatggcttgattaatcagacccttgcagctggtcgagcctcggctcttatactcaacaccttcgacgaactcgaagctccatttctgacccatctctcttccattttcaacaaaatctacaccattggacccctccatgctctgtccaaatcaaggctcggcgactcctcctcctccgcttctgccctctccggattctggaaagaggatagagcctgcatgtcctggctcgactgtcagccgccgaggtctgtggttttcgtcagtttcgggagtacgatgaagatgaaagccgatgaattgagagagttctggtatgggttggtgagcagcgggaaaccgttcctctgcgtgttgagatccgacgttgtttccggcggagaagcggcggaattgatcgaacagatggcggaggaggagggagctggagggaagctgggaatggtagtggagtgggcagcgcaagagaaggtcctgagccaccctgccgtcggtgggtttttgacgcactgcgggtggaactcaacggtggaaagcattgccgcgggagttccgatgatgtgctggccgattctcggcgaccaacccagcaacgccacttggatcgacagagtgtggaaaattggggttgaaaggaacaatcgtgaatgggacaggttgacggtggagaagatggtgagagcattgatggaaggccaaaagagagtggagattcagagatcaatggagaagctgtcaaagttggcaaatgagaaggttgtcaggggtgggttgtcttttgataacttggaagttctcgttgaagacatcaaaaaattgaaaccatataaattt

SEQ ID NO: 6 - DNA, 215 bp, Arabidopsis thaliana
ccaacactcgaatccccacccgttggaccaaacccggctcattaagcgtcggttcagatttatttcctttatttaaaaaaaaggaaagggtaaaaaatagaaaattggaaacagttaaagcccaaaattgtaatttaccgagaattgtaaatttacctgaaaaccctacgctatagtttcgactataaataccaaacttaggacctcacttcaga

SEQ ID NO: 7 - DNA, 585 bp, Figwort mosaic virus and Peanut Chlortic Streak Virus
gtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccagagtttttacttcggacagtcaaaaatgagtttaacttctcagccgaggtaaaacaagaaatatgcttacgtctacagagggatttctctgaagatcatgtttgccagctatgcgaacaatcatcgggagatcctgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtgcatccgctttgcctataaataagttttagtttgtattgatcgacacgatcgagaagacacggc

SEQ ID NO: 8 - DNA, 541 bp, Cassava vein mosaic virus
cagaaggtaattatccaagatgtagcatcaagaatccaatgtttacgggaaaaactatggaagtattatgtgaactcagcaagaagcagatcaatatgcggcacatattcaacctatgttcaaaaatgaagaatgtacagatacaagatcctatactgccagaatacgaagaagaatacatagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgacacgctggaattctagtatactaaac

SEQ ID NO: 9 - DNA, 500 bp, Horseradish Latent Virus
tggtagactatgaaacactagtctactcaaagaacttgaagaagacgactcaggaagacaggagcgtcatcaacaagtttcagcaaaagctgattagtggaaaaatccttggattccactctccagcaatctgccagcacataaaggtgacagcagaaaaagaagattgtgactaccactgcaatcagtgcgaatcttcaaaaggaaaggctatcgtttgcgataagcctgccgacagtggtccagccgacaatggagggccccagacacgtgaagacgcgtctgccgacagtgggtctaggacaacagacaccacgcactcagcaagtggatgaaataattcatctgctgacgtaagggatgacgatcaatcccactatcccaagacccttcacttctatataagtgaagttgcttcatttggagaaggcatctcgaaatctcaacacaactcgagctctcccttctctcttctttatctctctaaatgtgtgagtaga

SEQ ID NO: 10 - DNA, 242 bp, Peanut chlorotic streak caulimovirus
tgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtggatccgctttgcctataaataagttttagtttgtattgatcgacacgatcgagaagacacggcca

SEQ ID NO: 11 - DNA, 362 bp, Mirabilis mosaic virus
ttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaggatcccacaagaatttccttatataaggaacacaaatcagaaggaagagatcaatcgaaatcaaaatcggaatcgaaatcaaaatcggaatcgaaatctctcatct

SEQ ID NO: 12 - DNA, 677 bp, Cauliflower mosaic virus
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 13 - DNA, 425 bp, Cauliflower mosaic virus
acatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 14 - DNA, 427 bp, Cestrum yellow leaf curling virus
ctggcagacaaagtggcagacatactgtcccacaaatgaagatggaatctgtaaaagaaagcgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagacacgctggaaatctagtatactaaac

SEQ ID NO: 15 - DNA, 465 bp, Dahlia Mosaic Virus
caatcctcctcaggaaatgaaggattcaggagatcatctctatcaacttgctcaagtaaggacaaacgggttcacccggatcctccagaagacccagtctatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatgaggaaaaagacgtcagcagtgacgaacaagggcctgaagacttgcctatataatggcattcacccctcagttgaagagc

SEQ ID NO: 16 - DNA, 362 bp, Mirabilis mosaic virus; identical to SEQ ID NO: 11

SEQ ID NO: 17 - DNA, 382 bp, Figwort mosaic virus
cagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagcccgactcgaacatcttgaaggtgtacaaaacgttttagcagattgcctcacgagagattttaatgcttaaaacgtaagcgctgacgtatgatttcaaaaaacgcagctataaaagaagccctccagcttcaaagttttcatcaacacaaattctaaaaacaaaattttttagagagggggagtg

SEQ ID NO: 18 - DNA, 30 bp, Artificial Sequence
  Synthetic protein tag MYC
gaacagaagctcatcagcgaggaggatctg

SEQ ID NO: 19 - DNA, 33 bp, Artificial Sequence
  Synthetic protein tag HSV
caaccggaactggcacctgaagatccggaagat

SEQ ID NO: 20 - DNA, 24 bp, Artificial Sequence
  Synthetic protein tag FLAG
gactacaaagatgacgatgataag

SEQ ID NO: 21 - DNA, 27 bp, Artificial Sequence
  Synthetic protein tag HA
tacccgtatgacgtaccggattacgct

SEQ ID NO: 22 - DNA, 43 bp, Artificial Sequence
  Synthetic protein tag V5
ggcaaaccgatcccgaatccgcttctgggattagacagtactt

SEQ ID NO: 23 - DNA, 180 bp, Cauliflower mosaic virus
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcctgca

SEQ ID NO: 24 - DNA, 405 bp, Arabidopsis thaliana
tttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggccaac

SEQ ID NO: 25 - DNA, 486 bp, Arabidopsis thaliana
gaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctg

SEQ ID NO: 26 - DNA, 561 bp, Pisum sativum
gtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcaacatttattcactttcctttctgcaattttcaaatcatgtttgtagtttttattggacttgatgaatctctcttaccaccaaaatagaacattttttcttgatataccagaatctcaatatttgtctgataattattctagctgca

SEQ ID NO: 27 - DNA, 295 bp, Pisum sativum
attatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaac

SEQ ID NO: 28 - DNA, 1029 bp, Pisum sativum
atgtccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatctgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 29 - DNA, 792 bp, Aequorea victoria
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtactcagacctcgaactcaagctgcgtattctccagtcgacggttccgcgggcccgtgatccaccggatctcgataac

SEQ ID NO: 30 - DNA, 795 bp, Klebsiella pneumoniae
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 31 - DNA, 711 bp, Discosoma sp
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag